(19) United States
(12) Patent Application Publication
(10) Pub. No.: US 2022/0202348 A1
(43) Pub. Date: Jun. 30, 2022

(54) IMPLEMENTING BRAIN EMULATION NEURAL NETWORKS ON USER DEVICES

(71) Applicant: X Development LLC, Mountain View, CA (US)

(72) Inventors: Sarah Ann Laszlo, Mountain View, CA (US); David Andre, San Francisco, CA (US); Doris Tang, San Francisco, CA (US); Farooq Ahmad, Shoreline, WA (US)

(21) Appl. No.: 17/139,144

(22) Filed: Dec. 31, 2020

(51) Int. Cl. A61B 5/00; G06N 3/04; G06N 3/08

(52) U.S. Cl. CPC A61B 5/4064; G06N 3/0454; A61B 2503/40; A61B 5/7475; A61B 5/7267; G06N 3/08

(57) ABSTRACT

Methods, systems, and apparatus, including computer programs encoded on a computer storage medium, for implementing brain emulation neural networks on user devices. One of the methods includes obtaining, by a first component of a user device, a network input; processing, by the first component of the user device, the network input using an artificial neural network to generate a network output, wherein the artificial neural network has a network architecture that has been determined according to a synaptic connectivity graph, wherein the synaptic connectivity graph represents synaptic connectivity between neurons in a brain of a biological organism; and providing the network output for use by one or more second components of the user device.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of computational units to generate an output for a received input. For example, a deep neural network is a deep machine learning model that includes an output layer and one or more hidden layers that each apply a non-linear transformation to a received input to generate an output.

### SUMMARY

This specification describes systems implemented as computer programs on one or more computers in one or more locations for implementing, on a user device of a user, a neural network that includes a brain emulation neural network having a network architecture specified by a synaptic connectivity graph. In some implementations, the parameter values of the neural network can be updated after the neural network has been deployed onto the user device. The user device can be any appropriate device, e.g., a mobile phone or tablet, a laptop or desktop, a scientific field device, or an autonomous vehicle or drone.

A synaptic connectivity graph refers to a graph representing the structure of synaptic connections between neurons in the brain of a biological organism, e.g., a fly. For example, the synaptic connectivity graph can be generated by processing a synaptic resolution image of the brain of a biological organism. For convenience, throughout this specification, a neural network having an architecture specified by a synaptic connectivity graph may be referred to as a “brain emulation” neural network. Identifying an artificial neural network as a “brain emulation” neural network is intended only to conveniently distinguish such neural networks from other neural networks (e.g., with hand-engineered architectures), and should not be interpreted as limiting the nature of the operations that can be performed by the neural network or otherwise implicitly characterizing the neural network.

Particular embodiments of the subject matter described in this specification can be implemented so as to realize one or more of the following advantages.

The systems described in this specification can implement a brain emulation neural network having an architecture specified by a synaptic connectivity graph derived from a synaptic resolution image of the brain of a biological organism, or an image of a portion of the brain of the biological organisms, e.g., a ganglion or other neural cortex. The brains of biological organisms may be adapted by evolutionary pressures to be effective at solving certain tasks, e.g., classifying objects or generating robust object representations, and brain emulation neural networks can share this capacity to effectively solve tasks. In particular, compared to other neural networks, e.g., with manually specified neural network architectures, brain emulation neural networks can require less training data, fewer training iterations, or both, to effectively solve certain tasks. Moreover, brain emulation neural networks can perform certain machine learning tasks more effectively, e.g., with higher accuracy, than other neural networks.

The systems described in this specification can process a synaptic connectivity graph corresponding to a brain to select for neural populations with a particular function (e.g., sensor function, memory function, executive, and the like). In this specification, neurons that have the same function are referred to as being neurons with the same neuronal “type”. In particular, features can be computed for each node in the graph (e.g., the path length corresponding to the node and the number of edges connected to the node), and the node features can be used to classify certain nodes as corresponding to a particular type of function, i.e. to a particular type of neuron in the brain. A sub-graph of the overall graph corresponding to neurons that are predicted to be of a certain type can be identified, and a brain emulation neural network can be implemented with an architecture specified by the sub-graph, i.e., rather than the entire graph. Implementing a brain emulation neural network with an architecture specified by a sub-graph corresponding to neurons of a certain type can enable the brain emulation neural network to perform certain tasks more effectively while consuming fewer computational resources (e.g. memory and computing power). In one example, the brain emulation neural network can be configured to perform image processing tasks, and the architecture of the brain emulation neural network can be specified by a sub-graph corresponding to only the visual system of the brain (i.e., to visual system neurons). In another example, the brain emulation neural network can be configured to perform audio processing tasks, and the architecture of the brain emulation neural network can be specified by a sub-graph corresponding to only the audio system of the brain (i.e., to audio system neurons).

The systems described in this specification can use a brain emulation neural network in reservoir computing applications. In particular, a “reservoir computing” neural network can include a brain emulation subnetwork and one or more trained subnetworks. During training of the reservoir computing neural network, only the weights of the trained subnetworks are trained, while the weights of the brain emulation neural network are considered static and are not trained. In some implementations, a brain emulation neural network can have a very large number of parameters and a highly recurrent architecture; therefore, training the parameters of the brain emulation neural network can be computationally-intensive and prone to failure, e.g., as a result of the model parameter values of the brain emulation neural network oscillating rather than converging to fixed values. The reservoir computing neural network described in this specification can harness the capacity of the brain emulation neural network, e.g., to generate representations that are effective for solving tasks, without requiring the brain emulation neural network to be trained.

As described in this specification, brain emulation neural networks can achieve a higher performance (e.g., in terms of prediction accuracy), than other neural networks of an equivalent size (e.g., in terms of number of parameters). Put another way, brain emulation neural networks that have a relatively small size (e.g., 100 parameters) can achieve comparable performance with other neural networks that are much larger (e.g., thousands or millions of parameters).

Therefore, using techniques described in this specification, a system can implement a highly efficient and low-latency neural network for processing network inputs on user devices.

In some implementations described herein, a brain emulation neural network can operate at a fixed precision that is significantly lower than the precision of other neural networks, while achieving a comparable or higher performance. For example, the brain emulation neural network can operate at a 2-bit or 4-bit precision, while typical neural networks may operate at 32-bit precision. In some implementations described herein, the operations of the brain emulation neural network can be executed by an analog circuit; in these implementations, the bit precision can be expressed as a signal-to-noise ratio (SNR), where the SNR is proportional to 2n, where n is the bit precision if the neural network were executed digitally.

Operating at a lower bit precision allows the brain emulation neural network to consume significantly fewer computational resources, and generate network outputs significantly faster, than other neural networks. Furthermore, the memory cost of storing the parameter values of the brain emulation neural network is significantly reduced. As a particular example, the parameter values of a brain emulation neural network can be stored using just a few megabytes, e.g., 2, 5, 10, or 100 megabytes, while the parameter values for a typical neural network might require tens or hundreds of gigabytes to be stored.

The systems described in this specification can design an analog circuit that is configured to execute the operations of a neural network that includes a brain emulation neural network, and deploy the analog circuit onto a user device. Typically, an analog circuit can execute the operations of the neural network in less time, and using less energy, than if the operations were executed digitally, e.g., by the standard, non-dedicated processor of the user device. The specific structure of a brain emulation neural network can also lend itself particularly well to analog execution, as described in more detail below. For example, the analog circuit can execute the operations of a brain emulation neural network using significantly less power than if the operations were executed digitally, e.g., 2×, 10×, 100×, 1000×, or 10000× less power. As a particular example, the analog circuit might be able to execute the operations of the brain emulation neural network using only a few picojoules of energy. These efficiency gains can be especially important for use cases where the neural network continuously processes network inputs in the background, e.g., in an application that continuously processes audio data to determine whether a “wakeup” phrase has been spoken by a user. Furthermore, these efficiency gains can be especially important for use cases in which the user device is resource-constrained, ensuring that executing the operations of the neural network does not significantly reduce the battery life of the user device.

Generally, deploying a neural network that includes a brain emulation neural network directly onto a user device (whether digitally or as an analog circuit) decreases the time required before receiving a network output from the neural network compared to executing the neural network on the cloud, as the device can execute all operations of the neural network locally and does not need to communicate with the cloud.

In some implementations, the operations of a brain emulation neural network can be executed on a user device, ensuring the privacy of the user of the user device. In particular, a user input to the brain emulation neural network can be processed directly on the user device to generate a network output, as opposed to sending the user input to an external system, e.g., a cloud system, for processing. Thus, no personal information (e.g., audio or image data of the user) is exposed to an external system. Furthermore, in some implementations, the parameter values of the brain emulation neural network can be updated using a federated learning system, whereby training examples captured by respective user devices are used to improve the performance of the brain emulation neural network without ever leaving the user device, further ensuring user privacy.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example of generating a brain emulation neural network based on a synaptic resolution image of the brain of a biological organism.

FIG. 2 illustrates an example reservoir computing system.

FIG. 3 illustrates an example analog circuit design system.

FIG. 4 illustrates an example analog circuit deployment.

FIG. 5 illustrates an example brain emulation neural network inference system.

FIG. 6 illustrates an example federated learning system.

FIG. 7 shows an example data flow for generating a synaptic connectivity graph and a brain emulation neural network based on the brain of a biological organism.

FIG. 8 shows an example architecture mapping system.

FIG. 9 illustrates an example graph and an example sub-graph.

FIG. 10 is a flow diagram of an example process for designing and deploying an analog circuit configured to execute the operations of a brain emulation neural network.

FIG. 11 is a flow diagram of an example process for executing the operations of a brain emulation neural network on a user device.

FIG. 12 is a flow diagram of an example process for generating a brain emulation neural network.

FIG. 13 is a flow diagram of an example process for determining an artificial neural network architecture corresponding to a sub-graph of a synaptic connectivity graph.

FIG. 14 is a block diagram of an example computer system.

Like reference numbers and designations in the various drawings indicate like elements.

### DETAILED DESCRIPTION

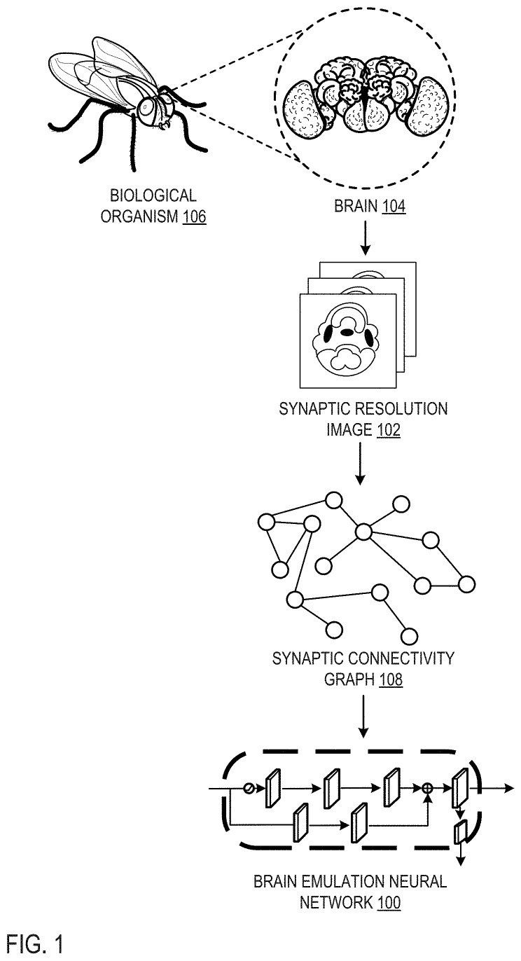

FIG. 1 illustrates an example of generating an artificial (i.e., computer implemented) brain emulation neural network 100 based on a synaptic resolution image 102 of the brain 104 of a biological organism 106, e.g., a fly. The synaptic resolution image 102 can be processed to generate a synaptic connectivity graph 108, e.g., where each node of the graph 108 corresponds to a neuron in the brain 104, and two nodes in the graph 108 are connected if the corresponding neurons in the brain 104 share a synaptic connection. The structure of the graph 108 can be used to specify the architecture of the brain emulation neural network 100. For example, each node of the graph 108 can mapped to an artificial neuron, a neural network layer, or a group of neural network layers in the brain emulation neural network 100. Further, each edge of the graph 108 can be mapped to a connection between artificial neurons, layers, or groups of layers in the brain emulation neural network 100. The brain 104 of the biological organism 106 can be adapted by evolutionary pressures to be effective at solving certain tasks, e.g., classifying objects or generating robust object representations, and the brain emulation neural network 100 can share this capacity to effectively solve tasks. These features and other features are described in more detail below.

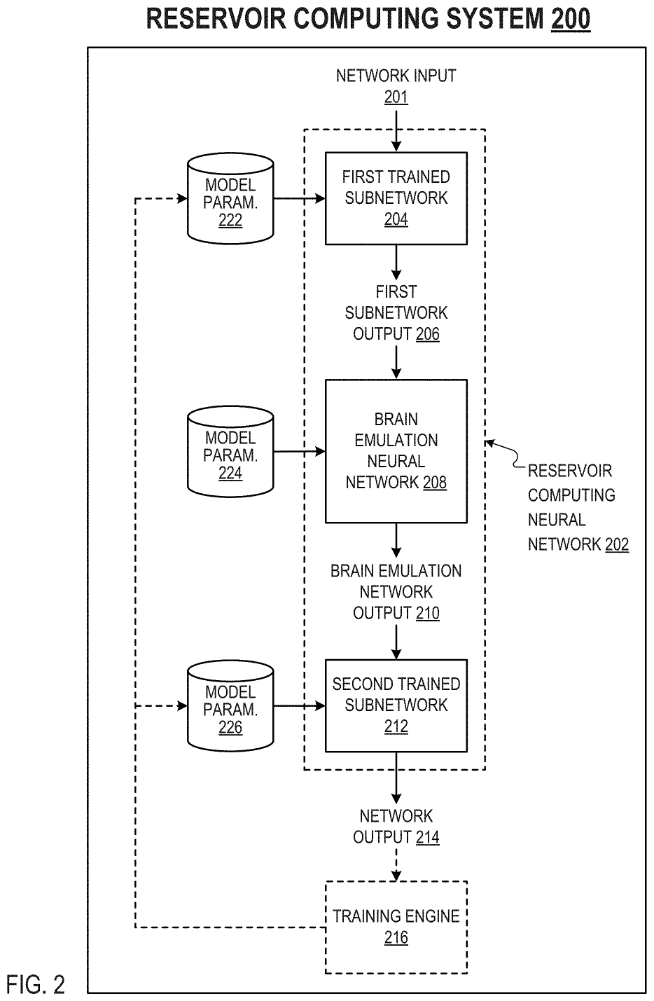

FIG. 2 shows an example reservoir computing system 200. The reservoir computing system 200 is an example of a system implemented as computer programs on one or more computers in one or more locations in which the systems, components, and techniques described below are implemented.

The reservoir computing system 200 includes a reservoir computing neural network 202 that has three subnetworks: (i) a first trained subnetwork 204 (ii) a brain emulation neural network 208, and (iii) a second trained subnetwork 212. The reservoir computing neural network 202 is configured to process a network input 201 to generate a network output 214. More specifically, the first trained subnetwork 204 is configured to process the network input 201 in accordance with a set of model parameters 222 of the first trained subnetwork 204 to generate a first subnetwork output 206. The brain emulation neural network 208 is configured to process the first subnetwork output 206 in accordance with a set of model parameters 224 of the brain emulation neural network 208 to generate a brain emulation network output 210. The second trained subnetwork 212 is configured to process the brain emulation network output 210 in accordance with a set of model parameters 226 of the second trained subnetwork 212 to generate the network output 214.

During training of the reservoir computing neural network 202, the parameter values of the one or more trained subnetworks 204 and 212 are trained, but the parameter values of the brain emulation neural network 208 are (optionally) static, i.e., not trained. Instead of being trained, the parameter values of the brain emulation neural network 208 can be determined from a synaptic connectivity graph, as will be described in more detail below. The reservoir computing neural network 202 facilitates application of the brain emulation neural network 208 to machine learning tasks by obviating the need to train the parameter values of the brain emulation neural network 208.

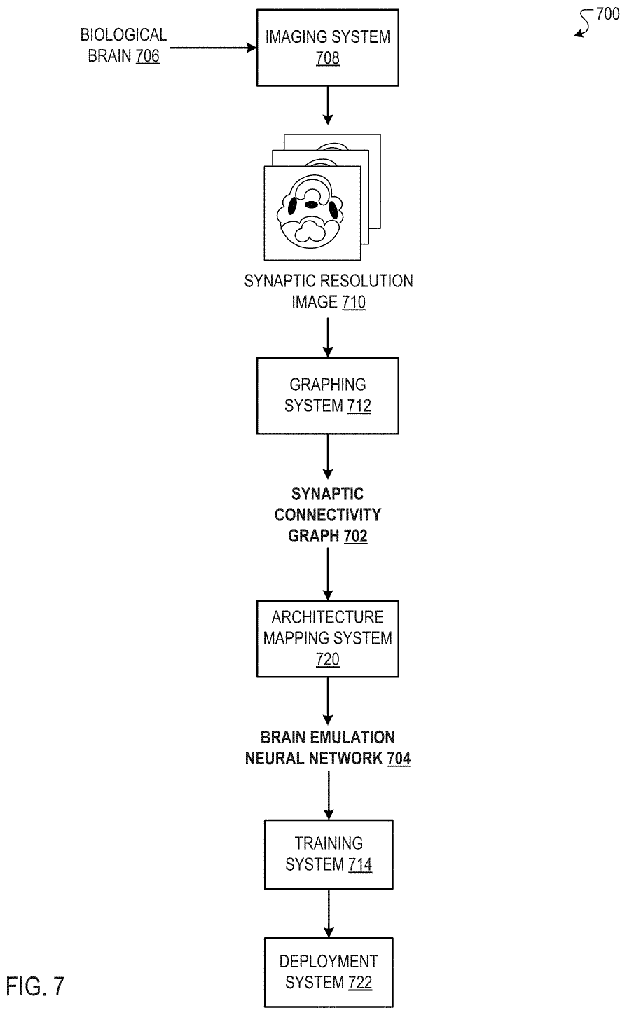

The brain emulation neural network 208 can have an architecture that is based on a graph representing synaptic connectivity between neurons in the brain of a biological organism. An example process for determining a network architecture using a synaptic connectivity graph is described below with respect to FIG. 7. The model parameters 224 can also be determined according to data characterizing the neurons in the brain of the biological organism; an example process for determining the model parameters of a brain emulation neural network is described below with respect to FIG. 7. In some cases, the architecture of the brain emulation neural network 208 can be specified by the synaptic connectivity between neurons of a particular type in the brain, e.g., neurons from the visual system or the olfactory system, as described above.

In some implementations, the first trained subnetwork 204 and/or the second trained subnetwork 212 can include only one or a few neural network layer (e.g., a single fully-connected layer) that processes the respective subnetwork input to generate the respective subnetwork output.

Although the reservoir computing neural network 202 depicted in FIG. 2 includes one trained subnetwork 204 before the brain emulation neural network 208 and one trained subnetwork 212 after the brain emulation neural network 208, in general the reservoir computing neural network 202 can include any number of trained subnetworks before and/or after the brain emulation neural network 208. For example, the reservoir computing neural network 202 can include zero, five, or ten trained subnetworks before the brain emulation neural network 208 and/or zero, five, or ten trained subnetworks after the brain emulation neural network 202. Generally there does not have to be the same number of trained subnetworks before and after the brain emulation neural network 202. In implementations where there are zero trained subnetworks before the brain emulation neural network 208, the brain emulation neural network can receive the network input 201 directly as input. In implementations where there are zero trained subnetworks after the brain emulation neural network 208, the brain emulation network output 210 can be the network output 214.

Although the reservoir computing neural network 202 depicted in FIG. 2 includes a single brain emulation neural network 208, in general the reservoir computing neural network 202 can include multiple brain emulation neural networks. In some implementations, each brain emulation neural network has the same set of model parameters 224. In some other implementations, each brain emulation neural network has a different set of model parameters 224.

In some implementations, the brain emulation neural network 208 has a recurrent neural network architecture. That is, the brain emulation neural network can process the first subnetwork output 206 multiple times at respective time steps. For example, the architecture of the brain emulation neural network 208 can include a sequence of components (e.g., artificial neurons, neural network layers, or groups of neural network layers) such that the architecture includes a connection from each component in the sequence to the next component, and the first and last components of the sequence are identical. In one example, two artificial neurons that are each directly connected to one another (i.e., where the first neuron provides its output the second neuron, and the second neuron provides its output to the first neuron) would form a recurrent loop. A recurrent brain emulation neural network can process a network input over multiple time steps to generate a respective brain emulation network output 210 of the network input at each time step. In particular, at each time step, the brain emulation neural network can process: (i) the network input, and (ii) any outputs generated by the brain emulation neural network 208 at the preceding time step, to generate the brain emulation network output 210 for the time step. The reservoir computing neural network 202 can provide the brain emulation network output 210 generated by the brain emulation neural network 208 at the final time step as the input to the second trained subnetwork 212. The number of time steps over which the brain emulation neural network 208 processes a network input can be a predetermined hyper-parameter of the reservoir computing system 200.

In some implementations, in addition to processing the brain emulation network output 210 generated by the output layer of the brain emulation neural network 208, the second trained subnetwork 212 can additionally process one or more intermediate outputs of the brain emulation neural network 208. An intermediate output refers to an output generated by a hidden artificial neuron of the brain emulation neural network, i.e., an artificial neuron that is not included in the input layer or the output layer of the brain emulation neural network.

The reservoir computing system 200 includes a training engine 216 that is configured to train the reservoir computing neural network 202. In some implementations, training the reservoir computing neural network 202 from end-to-end (i.e., training the model parameters 222 of the first trained subnetwork 204, the model parameters 224 of the brain emulation neural network 208, and the model parameters 226 of the second trained subnetwork 212) can be difficult due to the complexity and/or size of the architecture of the brain emulation neural network 208. In particular, the brain emulation neural network 208 can have a very large number of parameters and can have a highly recurrent architecture (i.e., an architecture that includes loops, as described above). Therefore, training the reservoir computing neural network 202 from end-to-end using machine learning training techniques can be computationally-intensive and the training can fail to converge, e.g., if the values of the model parameters of the reservoir computing neural network 202 oscillate rather than converge to fixed values. Even in cases where the training of the reservoir computing neural network 202 converges, the performance of the reservoir computing neural network 202 (e.g., measured by prediction accuracy) can fail to achieve an acceptable threshold. For example, the large number of model parameters of the reservoir computing neural network 202 can overfit a limited amount of training data.

Rather than training the entire reservoir computing neural network 202 from end-to-end, the training engine 216 can train only the model parameters 222 of the first trained subnetwork 204 and the model parameters 226 of the second trained subnetwork 212, while leaving the model parameters 224 of the brain emulation neural network 208 fixed during training. The model parameters 224 of the brain emulation neural network 208 can be determined before the training of the second trained subnetwork 212 based on the weight values of the edges in the synaptic connectivity graph, as described above. Optionally, the weight values of the edges in the synaptic connectivity graph can be transformed (e.g., by additive random noise) prior to being used for specifying model parameters 224 of the brain emulation neural network 208. This training procedure enables the reservoir computing neural network 202 to take advantage of the highly complex and non-linear behavior of the brain emulation neural network 208 in performing prediction tasks while obviating the challenges of training the brain emulation neural network 208.

The training engine 216 can train the reservoir computing neural network 202 on a set of training data over multiple training iterations. The training data can include a set of training examples, where each training example specifies: (i) a training network input, and (ii) a target network output that should be generated by the reservoir computing neural network 202 by processing the training network input.

At each training iteration, the training engine 216 can sample a batch of training examples from the training data, and process the training inputs specified by the training examples using the reservoir computing neural network 202 to generate corresponding network outputs 214. In particular, the reservoir computing neural network 202 processes each network input 201 using the current model parameter values 222 of the first trained subnetwork 204 to generate a first subnetwork output 206. The reservoir computing neural network 202 then processes the first subnetwork output 206 in accordance with the static model parameter values 224 of the brain emulation neural network 208 to generate a brain emulation network output 210. The reservoir computing neural network 202 then processes the brain emulation network output 210 using the current model parameter values 226 of the second trained subnetwork 212 to generate the network output 214. The training engine 216 adjusts the model parameters values 222 of the first trained subnetwork 204 and the model parameter values 226 of the second trained subnetwork 212 to optimize an objective function that measures a similarity between: (i) the network outputs 214 generated by the reservoir computing neural network 202, and (ii) the target network outputs specified by the training examples. The objective function can be, e.g., a cross-entropy objective function, a squared-error objective function, or any other appropriate objective function.

To optimize the objective function, the training engine 216 can determine gradients of the objective function with respect to the model parameters 222 of the first trained subnetwork 204 and the model parameters 226 of the second trained subnetwork 212, e.g., using backpropagation techniques. The training engine 216 can then use the gradients to adjust the model parameter values 226 of the prediction neural network, e.g., using any appropriate gradient descent optimization technique, e.g., an RMSprop or Adam gradient descent optimization technique. The training engine 216 can use any of a variety of regularization techniques during training of the reservoir computing neural network 202. For example, the training engine 216 can use a dropout regularization technique, such that certain artificial neurons of the brain emulation neural network are “dropped out” (e.g., by having their output set to zero) with a non-zero probability p>0 each time the brain emulation neural network processes a network input. Using the dropout regularization technique can improve the performance of the trained reservoir computing neural network 202, e.g., by reducing the likelihood of over-fitting. As another example, the training engine 216 can regularize the training of the reservoir computing neural network 202 by including a “penalty” term in the objective function that measures the magnitude of the model parameter values 226 of the second trained subnetwork 212. The penalty term can be, e.g., an L₁ or L₂ norm of the model parameter values 222 of the first trained subnetwork 204 and/or the model parameter values 226 of the second trained subnetwork 212.

In some cases, the values of the intermediate outputs of the brain emulation neural network 208 can have large magnitudes, e.g., as a result from the parameter values of the brain emulation neural network 208 being derived from the weight values of the edges of the synaptic connectivity graph rather than being trained. Therefore, to facilitate training of the reservoir computing neural network 202, batch normalization layers can be included between the layers of the brain emulation neural network 208, which can contribute to limiting the magnitudes of intermediate outputs generated by the brain emulation neural network. Alternatively or in combination, the activation functions of the neurons of the brain emulation neural network can be selected to have a limited range. For example, the activation functions of the neurons of the brain emulation neural network can be selected to be sigmoid activation functions with range given by [0,1].

The reservoir computing neural network 202 can be configured to perform any appropriate task. A few examples follow.

In one example, the reservoir computing neural network 202 can be configured to generate a classification output that classifies the network input into a predefined number of possible categories. For example, the network input can represent an image, each category can specify a type of object (e.g., person, vehicle, building, and the like), and the reservoir computing neural network 202 can classify an image into a category if the image depicts an object included in the category. As another example, the network input can represent an odor, each category can specify a type of odor (e.g., decomposing or not decomposing), and the reservoir computing neural network 202 can classify an odor into a category if the odor is of the type specified by the category.

In another example, the reservoir computing neural network 202 can be configured to generate an action selection output that can be used to select an action to be performed by an agent interacting with an environment. For example, the action selection output can specify a respective score for each action in a set of possible actions that can be performed by the agent, and the agent can select the action to be performed by sampling an action in accordance with the action scores. In one example, the agent can be a mechanical agent interacting with a real-world environment to perform a navigation task (e.g., reaching a goal location in the environment), and the actions performed by the agent cause the agent to navigate through the environment.

In another example, the reservoir computing neural network 202 can be configured to process sequences of network inputs 201, i.e., the reservoir computing neural network 202 can be a recurrent neural network. For example, each network input 201 can represent an audio example, and the reservoir computing neural network 202 can process the sequence of network inputs 201 to generate network outputs 214 representing predicted text samples that correspond to the audio samples. That is, the reservoir computing neural network 202 can be a “speech-to-text” neural network. As another example, each network input 201 can represent a text example, and the reservoir computing neural network 202 can process the sequence of network inputs 201 to generate network outputs 214 representing predicted audio samples that correspond to the text example. That is, the reservoir computing neural network 202 can be a “text-to-speech” neural network. As another example, each network input can represent a text example, and the reservoir computing neural network can generate network outputs 214 representing an output text example corresponding to the input text example. As a particular example, the output text samples can represent the same text as the input text samples in a different language (i.e., the reservoir computing neural network 202 can be a machine translation neural network). As another particular example, the output text samples can represent an answer to a question posed by the input text samples (i.e., the reservoir computing neural network 202 can be a question-answering neural network).

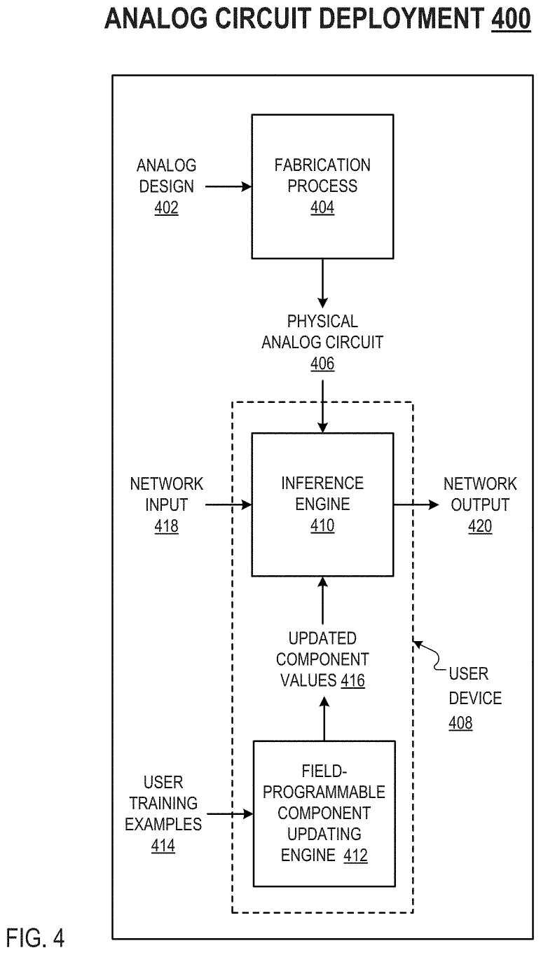

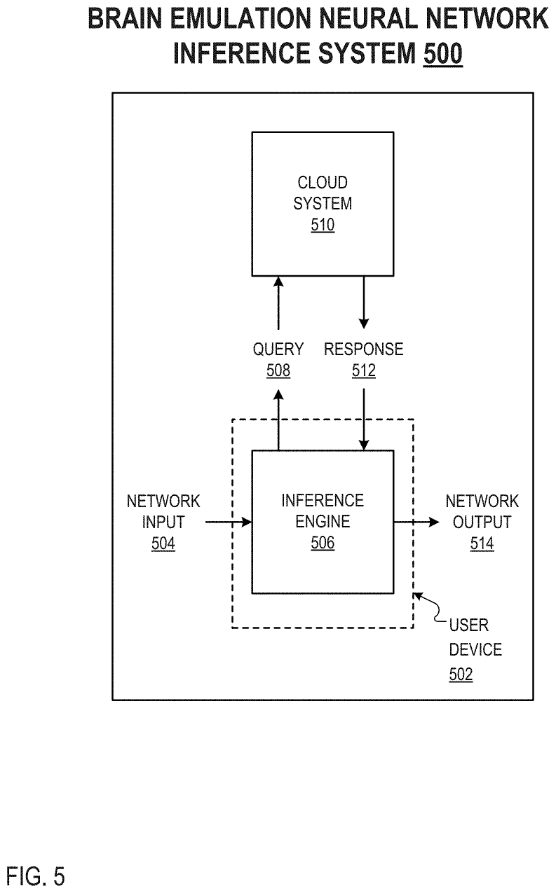

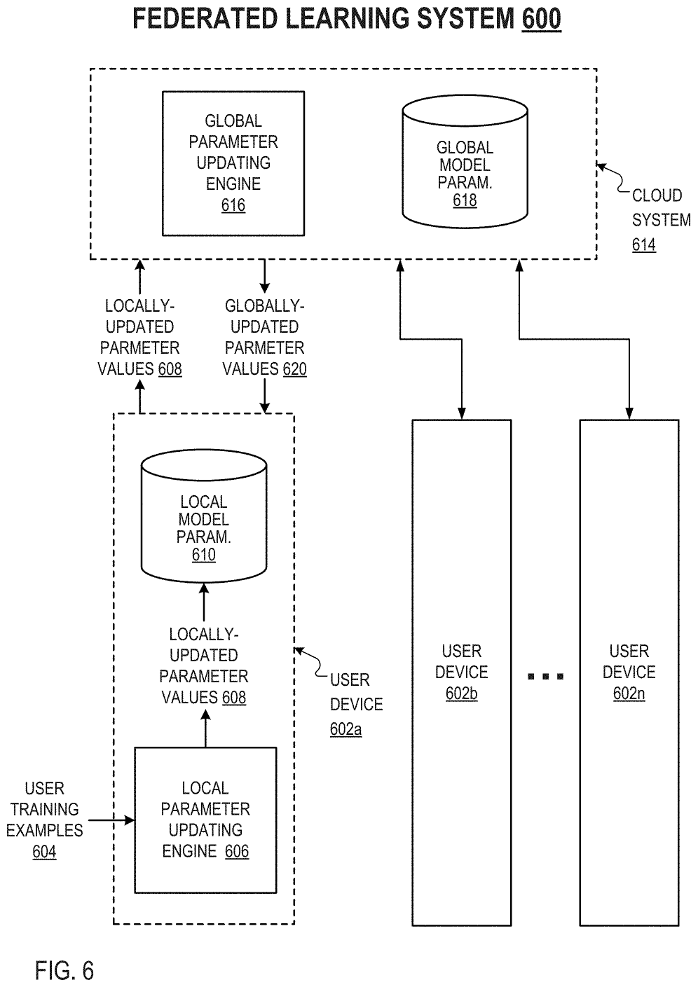

After training, the reservoir computing neural network 202 can be directly applied to perform prediction tasks. For example, the reservoir computing neural network 202 can be deployed onto a user device. Example processes for deploying a neural network that includes a brain emulation neural network onto a user device are discussed below with respect to FIG. 4, FIG. 5, and FIG. 6.

In some implementations, the reservoir computing neural network 202 can be deployed directly into resource-constrained environments (e.g., mobile devices). In some cases, reservoir computing neural networks 202 can perform at a high level, e.g., in terms of prediction accuracy, even with very few model parameters compared to other neural networks. For example, reservoir computing neural networks 202 as described in this specification that have, e.g., 100 or 1000 model parameters can achieve comparable performance to some other neural networks that have millions of model parameters. Thus, the reservoir computing neural network 202 can be implemented efficiently and with low latency on user devices.

In some other implementations, in order to further increase the computational and/or memory efficiency of the reservoir computing neural network 202, and/or to reduce the latency of the reservoir computing neural network 202, the reservoir computing neural network can be used to train a simpler “student” neural network as described above.

In some implementations, after the reservoir computing neural network 202 (or a student neural network determined according to the reservoir computing neural network 202, as described below with respect to FIG. 7) has been deployed onto a user device, some or all of the parameters of the reservoir computing neural network 202 can be further trained, i.e., “fine-tuned,” using new training examples obtained by the user device.

For example, some or all of the parameters can be fine-tuned using training example corresponding to the specific user of the user device, so that the reservoir neural network 202 can achieve a higher accuracy for inputs provided by the specific user. As a particular example, the reservoir computing neural network 202 can be configured to determine, from audio data captured of the environment surrounding the user device, whether a particular word or phrase, e.g., a wakeup phrase, has been spoken; in this example, the reservoir computing neural network 202 can be fine-tuned using training examples that include audio data of the specific user of the user device, in order to more accurately predict when the specific user speaks the word or phrase. For instance, the model parameters 222 of the first trained subnetwork 204 and/or the model parameters 226 of the second trained subnetwork 212 can be fine-tuned on the user device using new training exampled while the model parameters 224 of the brain emulation neural network 208 are held static, as described above.

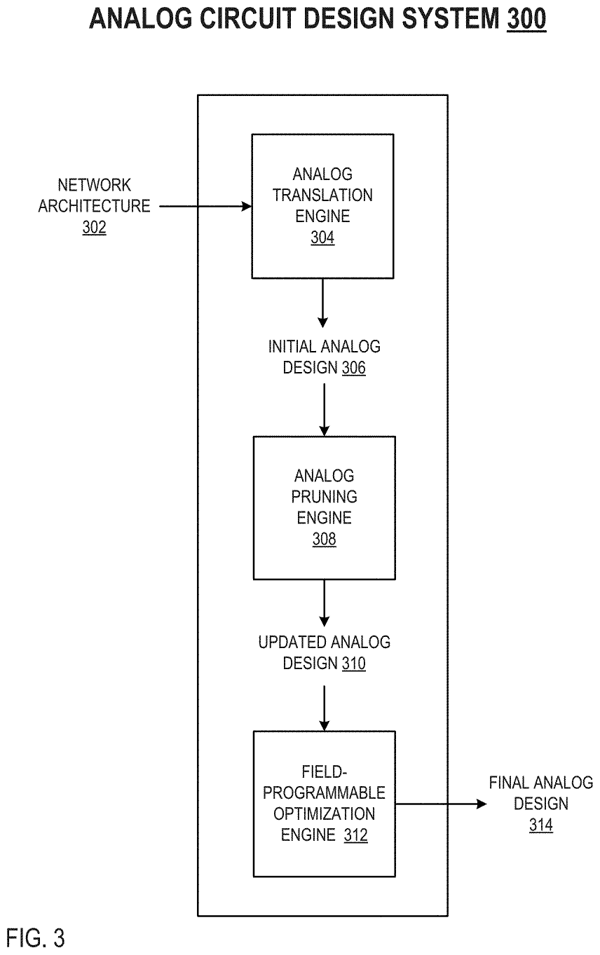

In some implementations, the operations of the reservoir computing neural network 202 can be executed using an analog circuit designed according to the network architecture of the reservoir computing neural network 202. Example processes for designing and deploying analog circuits based on brain emulation neural networks are discussed below with respect to FIG. 3 and FIG. 4.

FIG. 3 illustrates an example analog circuit design system 300. The analog circuit design system 300 is an example of a system implemented as computer programs on one or more computers in one or more locations in which the systems, components, and techniques described below are implemented.

The analog circuit design system 300 is configured to receive a network architecture 302, which is data representing the architecture of a neural network that includes a brain emulation neural network, and to process the network architecture 302 to generate a final analog design 314, which is data representing the design of an analog circuit that implements operations of the neural network represented by the network architecture 302. In this specification, an analog circuit is a physical electronic circuit (e.g., implemented on a chip) that supports a continuously-variable signal (e.g., as opposed to a strictly digital signal that can assume only two values, e.g., 0 and 1).

In this specification, an analog circuit design is a representation of an analog circuit that identifies the electronic elements of the analog circuit (e.g., the transistors, resistors, capacitors, etc. of the analog circuit) and the interconnections between the electronic elements. In some implementations, an analog circuit design is represented by a netlist. In this specification, a netlist is data describing the connectivity of an analog circuit, e.g., a list of the electronic elements of the analog circuit (e.g., resistors, capacitors, transistors, etc.) and a list of the nodes connecting the electronic elements. Instead or in addition to a netlist, an analog circuit design can include a representation of the physical layout of the analog circuit.

The brain emulation neural network can have an architecture that is based on a graph representing synaptic connectivity between neurons in the brain of a biological organism. For example, the brain emulation neural network can have been determined according to the process described below with respect to FIG. 7.

In some implementations, one or more subnetworks of the neural network are executed using an analog circuit, and one or more other subnetworks of the neural network are executed digitally. That is the analog circuit design system 300 can generate a final analog design 314 that represents the design of an analog circuit that implements operations of one or more subnetworks of the neural network represented by the network architecture 302, while the other subnetworks of the neural network represented by the network architecture 302 will be implemented digitally. As a particular example, if the neural network includes i) an untrained brain emulation neural network and ii) one or more trained subnetworks, then the analog circuit design system 300 can generate a final analog design 314 that implements the operations of the untrained brain emulation neural network, while the one or more trained subnetworks will be implemented digitally.

The analog circuit design system 300 includes an analog translation engine 304, an analog pruning engine 308, and a field-programmable optimization engine 312.

The analog translation engine 304 is configured to receive the network architecture 302 and to generate an initial analog design 306 that is data representing an initial design for the analog circuit that implements the operations of the neural network represented by the network architecture 302. In particular, each of the operations represented by the network architecture 302 are executed by the analog circuit represented by the initial analog design 306. That is, the analog translation engine 304 “translates” the operations of the network architecture 302 into analog versions of the operations in the initial analog design 306.

Generally, the analog translation engine 304 determines, for each artificial neuron of the neural network, one or more elements of the initial analog design 306 that will execute the operations of the artificial neuron in the analog circuit.

For example, the analog translation engine 304 can determine, for each artificial neuron of the neural network, multiple resistors that will execute the operations of the artificial neuron in the analog circuit. As a particular example, the initial analog design 306 can include on the order of 100 resistors for each artificial neuron of the neural network.

As another example, the analog translation engine 304 can determine, for each artificial neuron of the neural network, one or more transistors that will execute the operations of the artificial neuron in the analog circuit, optionally with corresponding linear circuit elements (e.g., resistors, capacitors, inductors, etc.). As a particular example, the initial analog design 306 can include a single transistor for each artificial neuron of the neural network.

As another example, the analog translation engine 304 can determine, for each artificial neuron of the neural network, a summing operational amplifier and activation non-linear circuit. In some cases, after a pruning process, this representation of an artificial neuron can be reduced to a single transistor, with necessary linear circuit elements (e.g., resistors, capacitors, inductors, etc.). Pruning is discussed in more detail below.

In this specification, a “component” of an analog circuit refers to the set of one or more electronic elements of the analog circuit that correspond to a particular artificial neuron of the neural network implemented by the analog circuit. For example, as described above, a component of an analog circuit that corresponds to a particular artificial neuron can include 100 resistors or a single transistor.

In some implementations, the neural network represented by the network architecture 302 can be a recurrent neural network, i.e., a neural network that processes a sequence of multiple network inputs at respective processing time steps. In some such implementations, the analog circuit designed by the analog circuit design system 300 can include one or more elements that maintain a hidden state of the recurrent neural network between processing time steps of the recurrent neural network. For example, the design 314 of the analog circuit can include one or more capacitance or delay lines that are configured to maintain the hidden state of the recurrent neural network.

The analog pruning engine 308 is configured to receive the initial analog design 306 and to generate an updated analog design 310 that is data representing an updated design for the analog circuit that implements the operations of the neural network represented by the network architecture 302. In particular, the analog pruning engine 308 can simplify the initial analog design 306 by removing, i.e., “pruning,” one or more electronic elements of the initial analog design 306 and/or one or more components of the initial analog design 306 in order to increase the efficiency and/or throughput of the analog circuit.

For example, the analog pruning engine 308 can select one or more electronic elements of the initial analog design 306 (e.g., one or more transistors and/or linear elements or wiring) and remove the one or more electronic elements of the initial analog design 306. For each selected electronic element, the analog pruning engine 308 can add an interconnect between i) each electronic element of the initial analog design 306 that had an incoming interconnect with the selected electronic element (i.e., each electronic element that passed a signal to the selected electronic element) and ii) each electronic element of the initial analog design 306 that has an outgoing interconnect with the selected electronic element (i.e., each electronic element to which the selected electronic element passed signal).

For example, the analog pruning engine 308 can select one or more artificial neurons of the neural network and, for each selected artificial neuron, remove the selected artificial neuron by determining i) each incoming connection for the selected artificial neuron (i.e., other artificial neurons in the neural network that pass data to the selected artificial neuron) and ii) each outgoing connection for the selected artificial neuron (i.e., other artificial neurons in the neural network to which the selected artificial neuron passes data). The analog pruning engine 308 can then add a connection in the neural network between i) the respective other artificial neuron corresponding to each incoming connection and ii) the respective other artificial neuron corresponding to each outgoing connection, thus removing the selected artificial neuron from the neural network. Then, the analog pruning engine can remove the component of the analog circuit that executes the operations of the selected artificial neuron.

In some implementations, the analog pruning engine 308 can obtain a training data set that includes multiple training examples, where each training example specifies: (i) a training input that can be processed by the neural network whose operations are implemented by the initial analog design 306, and (ii) a target output that should be generated by the neural network in response to processing the training input. The analog pruning engine 308 can use the training data set to simplify the initial analog design 306 in order to generate the updated analog design 310.

For example, the analog pruning engine 308 can determine a performance of the initial analog design 306, e.g., a prediction accuracy of the neural network as implemented by the initial analog design 306. In some implementations, the analog pruning engine 308 can obtain the performance of the initial analog design 306 from an external system, e.g., the training system that trained the neural network represented by the network architecture 302. Because, in some implementations, the operations of the initial analog design 306 can have a one-to-one correspondence with the operations of the network architecture 302, the performance of the initial analog design 306 can be the same as the performance of the network architecture 302. In some other implementations, the analog pruning engine 308 can determine the performance of the initial analog design 306 by simulating the analog circuit's processing of the training examples in the training data set according to the initial analog design 306, and determining a prediction accuracy of the network outputs generated by the simulated analog circuit using the respective target outputs.

For example, the analog circuit design system 300 can simulate the operations of the analog circuit using a SPICE (“Simulation Program with Integrated Circuit Emphasis”) simulator. As another example, the analog circuit design system 300 can simulate the operations of the analog circuit using a Xyce simulator.

The analog pruning engine 308 can determine one or more candidate updated analog designs, and determine the performance of each candidate updated analog design relative to the performance of the initial analog design 306. For example, for each of the one or more candidate updated analog designs, the analog pruning engine 308 can then remove one or more electronic elements and/or components from the initial analog design to generate the candidate updated analog design. The analog pruning engine 308 can then simulate the analog circuit's processing of the training examples in the training data set according to the candidate updated analog design in order to determine the performance of each candidate updated analog design on the training data set. In implementations in which the analog pruning engine 308 generates multiple candidate updated analog designs, the analog pruning engine 308 can then select one of the candidate updated analog designs to be the output updated analog design 310 according to the respective performances.

As a particular example, the analog pruning engine 308 can generate N candidate updated analog designs, where Nis a predetermined integer greater than one, and select the candidate updated analog design with the highest performance.

As another particular example, the analog pruning engine 308 can iteratively generate and analyze candidate updated analog designs until the performance of a particular candidate updated analog design exceeds a predetermined threshold, and select the particular candidate updated analog design. For example, the threshold can be defined with respect to the performance of the initial analog design 306, e.g., 80%, 90%, or 95% of the performance of the initial analog design 306.

As another particular example, at each of multiple iterations, the analog pruning engine 308 can generate a new candidate updated analog design according to the determined performances of the previous candidate updated analog designs generate and analyzed at previous iterations.

For example, the analog pruning engine 308 can generate the updated analog design 310 by performing backward elimination on the initial analog design 306. That is, at each iteration, the analog pruning engine 308 can select a different electronic element or component of the initial analog design 306 and remove the electronic element or component from the initial analog design 306 to generate a new candidate updated analog design. The analog pruning engine 308 can determine the performance of the new candidate updated analog design, e.g., using the training data set. The analog pruning engine 308 can then determine whether to permanently remove the selected electronic element or component from the analog design according to the determined performance. For example, the analog pruning engine 308 can determine to permanently remove the selected electronic element or component if the performance of the new candidate updated analog design declined by less than a predetermined threshold amount relative to i) the performance of the initial analog design 306 or ii) the determined performance of a previous candidate updated analog design generated at a previous iteration. If the analog pruning engine 308 determines to permanently remove the selected electronic element or component, then at the next iteration the analog pruning engine 308 will generate a new candidate updated analog design that does not include the selected electronic element or component. If the analog pruning engine 308 determines not to permanently remove the selected electronic element or component, then at the next iteration the analog pruning engine 308 will generate a new candidate updated analog design that does include the selected electronic element or component.

In some implementations, by pruning the initial analog design 306, the analog pruning engine 308 can identify a translation from a network architecture to an analog circuit design that that performs better, e.g., is more efficient than, the translations that the analog translation engine 304 uses. The analog pruning engine 308 can then provide data characterizing the superior translation to the analog translation engine 304 to be used for future network architectures 302 received by the analog circuit design system 300. That is, when processing a subsequent network architecture 302, instead of generating an initial analog design 306 that is a literal translation of the network architecture 302, the analog translation engine 304 can take a “shortcut” by generating an initial analog design 306 that already prunes one or more electronic elements, as identified by the superior translation, thus improving the efficiency of the analog circuit design system 300.

In some implementations, the updated analog design 310 can be significantly simpler than the initial analog design 306. For example, the updated analog design 310 can have 10×, 100×, or 1000× fewer operations than the initial analog design 306. As a particular example, if the initial analog design 306 represents millions or billions of operations, then the updated analog design can represent merely tens of thousands or hundreds of thousands of operations. As another particular example, if the initial analog design 306 represents tens of thousands or hundreds of thousands of operations, then the updated analog design 310 can represent merely thousands or hundreds of operations.

In particular, the analog pruning engine 308 can determine the operations of the initial analog design 306 that have the least bearing on the performance of the initial analog design 306 and remove the determined operations to generate the updated analog circuit design 310, so that the performance of the updated analog circuit design 310 is still in an acceptable range relative to the performance of the initial analog design 306. Therefore, the execution of an analog circuit fabricated according to the updated analog design 310 can be significantly more efficient and have a significantly higher throughput than an analog circuit fabricated according to the initial analog design 306, without significantly decreasing the performance.

This improved efficiency and throughput represents an advantage of executing the operations of neural network using an analog circuit instead of executing the operations digitally. Typically, a digital neural network accelerator does not execute the operations of a sparse neural network (i.e., a neural network with sparse weight matrices) more quickly than a dense neural network (i.e., a neural network with dense weight matrices) of the same size. That is, when the operations of a neural network are executed digitally, there are no efficiency gains from removing artificial neurons from the neural network. Analog circuits, on the other hand, are ideal for executing sparse and low-connectivity neural network architectures, because each artificial neuron of the neural network can be executed by a respective physical component of the analog circuit, and so removing the artificial neuron from the neural network allows the analog circuit design system 300 to remove the physical component from the design of the analog circuit, improving the efficiency of the analog circuit.

Furthermore, implementing brain emulation neural networks using analog circuits can provide particularly strong throughput and efficiency improvements because the architecture of a brain emulation neural network, in some implementations, can be sparser than the architectures of other neural network. In other words, in some implementations, the specific structure of a brain emulation neural network, as determined according to a synaptic connectivity graph as described above, lends itself particularly well to analog execution. In particular, the operations of a neural network that includes irregular bits (which would severely complicate a layer-based digital computation) can be simply executed using an analog circuit.

After generating the updated analog design 310, the analog pruning engine 308 can provide the updated analog design to the field-programmable optimization engine 312. In some implementations, the analog circuit design system 300 does not include the analog pruning engine 308. That is, the analog circuit design system 300 does not prune the operations of the initial analog design 306, and provides the initial analog design 306 directly to the field-programmable optimization engine 312.

The field-programmable optimization engine 312 is configured to receive the updated analog design 310 and to generate the final analog design 314. In particular, the field-programmable optimization engine 312 can select one or more components of the updated analog design 310 (corresponding to respective artificial neurons of the neural network) that will be field-programmable.

A field-programmable component of an analog circuit is a component whose value can be modified after the analog circuit has been fabricated; e.g., the value can be modified after the analog circuit has been deployed on a user device “in the field.” For example, a field-programmable component can be a programmable resistor (e.g., a memristor) or a programmable capacitor (e.g., a varicap).

Thus, the final analog design 314 can include data identifying the one or more components of the analog circuit that will be field-programmable. For example, the analog circuit can be a field-programmable analog array (FPAA).

After the analog circuit has been deployed onto a user device, the user device can update the values of the selected field-programmable components of the analog circuit using user data captured by the user device. This process is described in more detail below with reference to FIG. 4.

In some implementations, each field-programmable component of the analog circuit includes one or more memristors that execute the operations of the field-programmable artificial neurons of the neural network.

In some implementations, the neural network includes i) an untrained brain emulation neural network and ii) one or more trained subnetworks. For example, the neural network can include a first trained subnetwork (e.g., the first trained subnetwork 204 depicted in FIG. 2) that includes one or more trained input neural network layers, and a second trained subnetwork (e.g., the second trained subnetwork 212 depicted in FIG. 2) that includes one or more trained output neural network layers. In these implementations, the field-programmable optimization engine 312 typically only selects components of the updated analog design 310 that correspond to artificial neurons of the trained subnetworks to be field-programmable. That is, the field-programmable optimization engine 312 does not select any components corresponding to artificial neurons of the brain emulation neural network to be field-programmable, because the artificial neurons of the brain emulation are not trained, having been determined according to a synaptic connectivity graph.

To select the one or more field-programmable components of the analog circuit, the field-programmable optimization engine 312 determines the one or more components of the updated analog design 310 that, when updated using training data that is user-specific, most improves the performance of the neural network.

The field-programmable optimization engine 312 can obtain a training data set that includes multiple training examples, where each training example specifies: (i) a training input that can be processed by the neural network whose operations are implemented by the updated analog design 310, and (ii) a target output that should be generated by the neural network in response to processing the training input. The field-programmable optimization engine 312 can use the training data set to select the one or more field-programmable components.

For example, the training data set can include multiple training examples that correspond to each of multiple different users. That is, for each training example corresponding to a particular user, the training input of the training example has been generated from a user input of the particular user or otherwise characterizes the particular user.

As a particular example, for each of multiple candidate components that might be selected by the field-programmable optimization engine 312 (e.g., for each component corresponding to an artificial neuron of a trained subnetwork of the neural network) and for each particular user of the multiple different users, the field-programmable optimization engine 312 can simulate the analog circuit's processing of the multiple different training examples corresponding to the user according to the updated analog design 310, generating a respective network output for each training example. The field-programmable optimization engine 312 can then determine an update to the value of the candidate component according to an error between the network outputs and the respective target outputs. The field-programmable optimization engine 312 can then determine an improvement to the performance of the neural network caused by the parameter update of the candidate component determined according to the training examples of the particular user.

The field-programmable optimization engine 312 can select one or more of the candidate components to be field-programmable according to the respective improvements to the performance of the neural network caused by the parameter updates of the respective candidate components determined according to the training examples of the respective different users. For example, for each candidate component, the field-programmable optimization engine 312 can determine an average improvement to the performance of the neural network caused by the parameter updates of the candidate component determined according to the respective different users. The field-programmable optimization engine 312 can then select the one or more candidate components with the highest corresponding average improvement.

In some implementations, the field-programmable optimization engine 312 selects a predetermined number of field-programmable components, e.g., the N candidate components with the highest corresponding average improvement. For example, the analog circuit design system 300 might be constrained to selecting at most N field-programmable components because it can be significantly more expensive to fabricate field-programmable components than fixed analog components. Furthermore, increasing the number of field-programmable components can require a more sophisticated process for updating the values of the components in the field, more extensive software or hardware for executing the updating process, and/or more training data and longer training times for updating the values.

In some other implementations, the field-programmable optimization engine 312 selects each candidate component that satisfies one or more conditions, e.g., each candidate component whose corresponding average improvement satisfies a predetermined threshold.

In some implementations, the analog pruning engine 308 and the field-programmable optimization engine 312 are the same system. That is, a single system can concurrently determine a first set of one or more components of the initial analog design 306 that are to be removed and a second set of one or more components of the initial analog design 306 that are to be field-programmable. For example, the system can concurrently determine the first and second sets of components by processing training examples, as described above.

In some other implementations, the analog circuit design system 300 does not include a field-programmable optimization engine 312. For example, the analog circuit design system 300 can be configured to generate final analog designs 314 that do not include any field-programmable components. In this example, the analog circuit design system 300 can output the updated analog design 310 as the final analog design 312. As another example, the analog circuit design system 300 can simply select each candidate component to be field-programmable, e.g., select each component that corresponds to a trained artificial neuron of the neural network.

After generating the final analog design 314, the analog circuit design system 300 can provide the final analog design 314 to a fabrication system for fabricating analog circuits according to the final analog design. The fabricated analog circuits can then be deployed onto user devices.

FIG. 4 illustrates an example analog circuit deployment 400. During the analog circuit deployment 400, an analog circuit design 402, which is data representing the design of an analog circuit that implements operations of a neural network that includes a brain emulation neural network, is deployed to a physical analog circuit 406 fabricated according to the analog circuit design 402 onto a user device 408. That is, the physical analog circuit 406 implements the operations of the analog circuit design 402, e.g., using discrete components on a circuit board, or as an analog chip, or a combination thereof. For example, the analog circuit design 402 can be generated by the analog circuit design system 300 depicted in FIG. 3.

As described above, the brain emulation neural network can have an architecture that is based on a graph representing synaptic connectivity between neurons in the brain of a biological organism. For example, the brain emulation neural network can have been determined according to the process described below with respect to FIG. 7.

To begin the analog circuit deployment 400, a manufacturing system executes a fabrication process 404 by receiving the analog circuit design 402 and fabricating the physical analog circuit 406 according to the analog circuit design 402. The physical analog circuit 406 can include a combination of board- and chip-level circuitry. That is, the manufacturing system physically manufactures the physical analog circuit 406. For example, the manufacturing system can include one or more semiconductor fabrication plants in respective locations in the world that manufacture electronic devices.

The physical analog circuit 406 can then be deployed onto the user device 408, where it is configured to execute the operations of the neural network. In particular, after being deployed onto the user device 408, the analog circuit 406 is a component of an inference engine 410 that is configured to receive a network input 418, process the network input 418 using the analog circuit 406 to generate a network output 420, and provide the network output to one or more other systems of the user device 408. An example inference system for a brain emulation neural network deployed onto a user device is described in more detail below with reference to FIG. 5.

In some implementations, the physical analog circuit 406 includes one or more components that are field-programmable, i.e., whose values can be updated after the analog circuit 406 is deployed onto the user device 408. In these implementations, the user device 408 includes a field-programmable component updating engine 412 that is configured to update the values of the field-programmable components of the analog circuit 406.

In particular, the field-programmable component updating engine 412 is configured to receive user training examples 414 and to use the user training examples 414 to update the values of the field-programmable components of the analog circuit 406. Each user training example can include (i) a training input and (ii) a target output.

Each user training example 414 has been generated from a user input of the user of the user device 408 or otherwise characterizes the user of the user device 408. For example, the neural network can be configured to process audio data, and the user training examples 414 can include audio data spoken by the user (or inputs generated from audio data spoken by the user, e.g., spectrograms). As a particular example, the neural network can be configured to predict whether the audio data includes a verbalization of a predefined work or phrase, e.g., a “wakeup” phrase of a user device 408. In some implementations, the user device 408 can prompt the user to provide one or more audio clips of the user speaking the wakeup phrase (e.g., by speaking into a microphone of the user device 408); the user device 408 can then generate the user training examples 414 using the audio clips.

As another example, the neural network can be configured to process image data (e.g., RGB image data or infrared image data), and the user training examples 414 can include images of the user (or inputs generated from images of the user), e.g., images of the user's face. As a particular example, the neural network can be configured to predict whether an image depicts the face of the user (e.g., to verify the identity of the user). In some implementations, the user device 408 can prompt the user to provide one or more images of the user's face (e.g., using a camera of the user device 408); the user device 408 can then generate the user training examples 414 using the images.

As another example, the neural network can be configured to process health data, e.g., data captured by a wearable device, and the user training examples 414 can include health data of the user, e.g., health data captured by a wearable device (i.e., the user device 408 if the user device 408 is a wearable device, or another wearable device).

The field-programmable component updating engine 412 can process the user training examples 414 to generate updated values 416 of the field-programmable components. In some implementations, the field-programmable component updating engine 412 processes the training inputs of the user training examples 414 using the physical analog circuit 406 to generate respective network outputs. In some other implementations, the field-programmable component updating engine 412 simulates the processing of the training inputs by the physical analog circuit to generate the respective network outputs. The field-programmable component updating engine 412 can then determine an error between the network outputs and the respective target outputs, and use the determined error to generate the updated values 416 of the field-programmable components of the physical analog circuit 406, e.g., using backpropagation and stochastic gradient descent.

One or more of the field-programmable components of the physical analog circuit 406 might precede, in the architecture of the neural network, one or more other components that correspond to the brain emulation neural network. For example, the one or more field-programmable components can correspond to artificial neurons of an input neural network layer that precedes the brain emulation neural network in the architecture of the neural network. In some implementations, the brain emulation neural network has an irregular structure that does not allow the field-programmable component updating engine 412 to perform backpropagation through the brain emulation neural network (e.g., a structure that cannot be represented as an invertible matrix). Thus, the field-programmable component updating engine 412 cannot generate the updated values 416 for the one or more field-programmable components by analytically backpropagating the determined error to the artificial neurons of the neural network corresponding to the field-programmable components. In some such implementations, the field-programmable component updating engine 412 numerically determines the gradients of the artificial neurons of the neural network corresponding to the field-programmable components with respect to the error. The field-programmable component updating engine 412 can then generate the updated values 416 for the one or more field-programmable components using the numerically-determined gradients, e.g., using stochastic gradient descent.

After the field-programmable component updating engine 412 generates the updated values 416 for the field-programmable components, the engine 412 can provide the updated values 416 to the inference engine 410, which can trim the values of the field-programmable components of the physical analog circuit 406 to reflect the received updated values 416. In this specification, “trimming” a component of an analog circuit is the process of physically configuring the value of the component. Trimming a component can also be referred to as electronically adjusting or programming the component.

For example, the inference engine 410 can trim the values of the field-programmable components using resistive random access memory (RRAM). As another example, the inference engine 410 can trim the values of the field-programmable components using non-volatile analog memory (e.g., memristors). As a particular example, the inference engine 410 can trim the values of the field-programmable components using conductive-bridge random-access memory (CBRAM), e.g., by providing higher voltages to alter the distribution of conductors in the CBRAM.

After the physical analog circuit 406 is deployed onto the user device 408, the analog circuit 406 can execute the operations of the neural network in less time, and using less energy, than if the operations were executed digitally.

These efficiency gains can be particularly advantageous for use cases where the neural network continuously (or very frequently) processes network inputs 418 in the background of the user device 406. In particular, the reduced energy costs of the physical analog circuit 406 can ensure that continuously executing the neural network does not significantly reduce the battery life of the user device 408.

For example, as described above, the neural network can be configured to continuously process audio data (or network inputs 418 generated from audio data) captured by the user device 408 and to generate network outputs 420 that represent a prediction of whether the input audio data is a verbalization of a predefined work or phrase.

As another example, the neural network can be configured to iteratively process network inputs 418 characterizing the face of the user of the user device 408 (e.g., network inputs 418 that include one or more of: infrared images of the face of the user, lidar data representing the face of the user, or a depth map of the face of the user) in order to verify the identity of the user, e.g., in order to unlock the user device or to process a payment.

As another example, the neural network can be configured to continuously process health data of the user captured by user device 408 and to generate network outputs 420 that characterize a prediction of the health of the user. As a particular example, the neural network can be configured to perform sleep staging using the health data of the user, or to generate a prediction of whether the user is experiencing a medical emergency, e.g., a heart arrhythmia.

As another example, the user device 408 can be a drone and the neural network can be configured to continuously process network inputs 418 representing the current state of the drone in order to stabilize the flight of the drone.

The efficiency gains of the physical analog circuit 406 can also be particularly advantageous for use cases in which the user device 406 is resource-constrained.

For example, the user device 408 can be a scientific field device that is used in environments that do not provide access to a power source, requiring the user device 408 to execute the neural network without significantly draining the battery of the user device 408. As a particular example, the neural network can be configured for a computational agriculture use case, where a user captures data, e.g., images, representing the current state of crops in the field and processes the data using the neural network to generate a prediction about the crops, e.g., the health of the crops.

As another example, the user device 408 can be a long-term device that is installed in a location, over the course of multiple days, months, or years, continuously captures data and processes the data using the neural network. For example, the user device 408 can be configured to monitor the ambient environment in the location, e.g., a warehouse or other facility, and to and notify a user if an issue is detected.

FIG. 5 illustrates an example brain emulation neural network inference system 500. The brain emulation neural network inference system 500 is an example of a system implemented as computer programs on one or more computers in one or more locations in which the systems, components, and techniques described below are implemented.

The brain emulation neural network inference system 500 can implement the operations of a neural network that includes a brain emulation neural network on a user device 502. As described above, the brain emulation neural network can have an architecture that is based on a graph representing synaptic connectivity between neurons in the brain of a biological organism. For example, the brain emulation neural network can have been determined according to the process described below with respect to FIG. 7.

In particular, the brain emulation neural network inference system 500 executes the operations of the neural network using an inference engine 506. The inference engine 506 is configured to receive a network input 504 and to process the network input 504 using the neural network to generate a network output 514. In some implementations, the inference engine 506 executes the operations of the neural network using an analog circuit designed to implement the neural network, e.g., an analog circuit designed using the analog circuit design system 300 depicted in FIG. 3. In some other implementations, the inference engine 506 executes the operations of the neural network digitally.

In some implementations, the parameters of the neural network are maintained at a fixed precision, and the inference engine 506 operates at the fixed precision. The fixed precision can be low relative to the typical precision of a neural network. For example, while a typical neural network can operate at 32-bit precision, the neural network that includes the brain emulation neural network can operate at 1-bit, 2-bit, 3-bit, 4-bit, or 8-bit fixed precision. In some such implementations, the network inputs 504 received by the inference engine 506 are expressed at a higher (e.g., 32-bit) precision than the fixed precision of the neural network, and so the inference engine 506 quantizes the network input 504 to match the fixed precision. In these implementations, the neural network that includes the brain emulation neural network can operate at the lower fixed precision and still achieve a comparable accuracy to other neural networks of a similar size. That is, brain emulation neural networks, determined according to a synaptic connectivity graph, enable the inference engine 506 to achieve a comparable performance to other neural networks while operating at a significantly lower fixed precision, thus increasing the efficiency and decreasing the latency of the inference engine 506.

After generating the network output 514, the inference engine 506 can provide the network output 514 to one or more external systems of the user device 502, e.g., the system that submitted the network input 504.

Instead of or in addition to outputting the generated network output 514, the inference engine 506 can determine that the network output 514 represents a query 508 to a cloud system 510. That is, the inference engine 506 can determine the query according to the network output 514.

The inference engine 506 can provide the query 508 to the cloud system 510, which processes the query 14 to determine a response 512. The cloud system 510 can then provide the response 512 to the inference engine 506. For example, the query 508 can be a query to a database system of the cloud system 510, and the cloud system 510 can retrieve the queried data from the database system and include the queried data in the response 512. As another example, the query 508 can be a query to retrieve one or more webpages from the Internet or an intranet, and the cloud system 510 can retrieve the requested webpages and include the requested webpages in the response 512. As a particular example, the query 508 can include a query to a search engine, and the cloud system 510 can obtains the results of the query to the search engine, e.g., one or more webpages that match the parameters of the query, and include the results in the response 512.

In some implementations, the inference engine 506 does not provide the network input 504 or any other data of the user device 502 to the cloud system 510 when submitting the query 508. That is, the inference engine 506 can process the network input 504 on the user device 502 to determine the parameters of the query 508, and then submit only the determined query to the cloud system 510.

Thus, by executing the operations of the neural network on the user device 502, the inference engine 506 can protect the privacy of the user of the user device 502. For example, the network input 504 can include personal information of the user, e.g., audio data of the user, images of the user, health data of the user, etc. By processing the network inputs 506 locally on the user device 502, the inference engine 506 ensures that no personal information is sent to the cloud system 510. In some implementations, the local execution of the neural network is enabled by the fact that the brain emulation neural network can be executed at a significantly lower precision, and/or can include significantly fewer parameters, and still achieve a comparable performance to other neural networks, thus reducing the computational, memory, and/or energy cost of executing the brain emulation neural network locally. This represents an advantage over other systems that execute inference calls of a neural network on an external system, requiring user devices to send network inputs that might include personal information to the external system in order to process the network inputs using the neural networks.

As a particular illustrative example, the inference engine 506 can continuously process audio data captured from the environment of the user device 502 to determine whether the user has spoken a “wakeup” phrase that causes the user device 502 to turn on in response to a verbal prompt from the user. That is, the network output 514 can be a prediction of whether a verbalization of the wakeup phrase is represented by the network input 504. In particular, the inference engine 506 processes the network inputs 504 on the user device 502 so that the audio data does not leave the user device 502. In some existing systems, a user device must continuously send audio data to an external system, which executes processes the audio data using a neural network and sends back to the user device a prediction of whether the audio data represents a verbalization of the wakeup phrase. Thus, in these existing systems, the user device is continuously recording its environment and sending the audio recordings to an external system.

Continuing the above illustrative example, in some implementations of the inference system 500 in which the inference engine 506 executes the neural network digitally, the inference engine 506 continuously records the audio data of the environment of the user device 502 into a “scratchpad” memory and, after processing the network input corresponding to the audio data, immediately deletes the audio data from the scratchpad memory. In some implementations in which the inference engine 506 executes the neural network using an analog circuit, the analog circuit does not digitally record the audio data at all, instead continuously storing the audio data in capacitors of the analog circuit.

If the audio data represented by the network input 504 did include a verbalization of the wakeup phrase, and the audio data further included a verbalized request by the user to send a query to the cloud system 510 (e.g., a request to query a search engine), then the inference engine 506 can generate the query 508 as described above and send the query 508, and not the audio data or any other personal information of the user.

The user device 502 can be any appropriate device, e.g., a mobile device such as a phone, tablet, or laptop. Some other examples follow.

The user device 502 can be a scientific field device, e.g., a computational agriculture device as described above. The execution of the neural network locally on the device 502 can be especially important for use cases where the user device 502 does not have network access, e.g., Internet access, in the field. Thus, the user does not need to capture data in the field that will be used to generate network inputs 504 and then return from the field to a location that has network access in order to upload the network inputs 504 to an external system that executes the neural network; rather, the user can process the network inputs 504 in the field directly on the device 502, allowing the user to review the corresponding network outputs 514 and receive immediate feedback.

The user device 502 can be an autonomous or semi-autonomous vehicle or drone. The smaller model size, lower fixed precision, and/or increased efficiency of some brain emulation neural networks as described above can allow the vehicle or drone to execute the neural network even when the vehicle or drone is resource-constrained. The higher throughput of some brain emulation neural networks can be especially important for time-sensitive tasks performed by the vehicle or drone, e.g., when a vehicle is processing sensor data using the neural network to determine whether the sensor data represents a pedestrian.

FIG. 6 illustrates an example federated learning system 600. The federated learning system 600 is an example of a system implemented as computer programs on one or more computers in one or more locations in which the systems, components, and techniques described below are implemented.

The federated learning system 600 includes a cloud system 614 and N user devices 602a-n. The federated system 600 is configured to update the parameters of a neural network that includes a brain emulation neural network using training examples gathered by each of the user devices 602a-n without sending any of the training examples to the cloud system 614, thus ensuring the privacy of the respective users of the user devices 602a-n.

As described above, the brain emulation neural network can have an architecture that is based on a graph representing synaptic connectivity between neurons in the brain of a biological organism. For example, the brain emulation neural network can have been determined according to the process described below with respect to FIG. 7.

In some implementations, the one or more user devices 602a-n execute the operations of the neural network using an analog circuit designed to implement the neural network, e.g., an analog circuit designed using the analog circuit design system 300 depicted in FIG. 3. Instead or in addition, one or more other user devices 602a-n can executes the operations of the neural network digitally.

Each user device 602a-n includes a local parameter updating engine 606 and a local model parameter store 610. For clarity, in FIG. 6 the local parameter updating engine 606 and local model parameter store 610 are only illustrated in the first user device 602a.

The local model parameter store 610 for each user device 602a-n is configured to store a respective local version of the current values for the parameters of the neural network. Initially, each local model parameter store 610 of the respective user devices 602a-n can store the same set of initial values for the parameters of the neural network.

The local parameter updating engine 606 of each user device 602a-n is configured to obtain user training examples 604 corresponding to the user of the respective user device 602a-n and to process the user training examples 604 to update the parameter values of the neural network, generating locally-updated parameter values 608. As described above, the local parameter updating engine 606 can update the parameter values according to an error between i) a target network output identified in a user training example 604 and ii) the network output generated by the neural network in response to processing the user training example 604. For example, the user can provide the target network outputs for each user training example 604. As a particular example, the local parameter updating engine 606 can provide, for each user training example 604, a prompt to the user identifying the user training example 604 (e.g., by displaying the prompt on a display of the user device 602a-n), and the user can submit a user input that identifies the target network output corresponding to the user training example 604.

In some implementations, the local parameter updating engine 606 can update the value for each parameter of the neural network. In some other implementations, the local parameter updating engine 606 only updates the value for a subset of the parameters of the neural network. For example, if the neural network includes i) a trained brain emulation neural network and ii) one or more trained subnetworks, the local parameter updating engine 606 can only update the parameters of the one or more trained subnetworks. As a particular example, if the neural network is implemented by an analog circuit, only a few of the parameters of the one or more trained subnetworks might be field-programmable, i.e., able to be updated.

The local parameter updating engine 606 can provide the locally-updated parameter values 608 to the local model parameter store 610, which can store the locally-updated parameter values 608 for future inference calls of the neural network on the respective device 602a-n.

Each user device 602a-n can also provide the respective locally-updated parameter values 608 to the cloud system 614. In particular, each user device 602a-n can provide only the locally-updated parameter values 612, and not the user training examples 604 or any other data of the user device 602a-n, to the cloud system 614, because the user training examples 604 might include personal information of the respective user of the user device 602a-n.

The cloud system 614 includes a global parameter updating engine 616 and a global model parameter store 618. The global model parameter store 618 is configured to store a global version of the current values for the parameters of the neural network. Initially, the global model parameter store 618 can store the same set of initial values for the parameters as the respective local model parameter stores 610.

The global parameter updating engine 616 is configured to obtain the respective locally-updated parameter values 608 from each of one or more user devices 602a-n and use the sets of locally updated parameter values 608 to determine an update to the parameter values of the neural network stored in the global model parameter store 618. The global parameter updating engine 616 can combine i) the current version of the parameter values stored in the global model parameter store 618 and ii) the one or more sets of locally-updated parameter values 608, in any appropriate way. For example, the global parameter updating engine 616 can determine a weighted mean of different versions of the parameter values. As a particular example, the global parameter updating engine can weight the version stored in the global model parameter store 618 more than each of the locally-updated versions 608. As another particular example, the global parameter updating engine 616 can weight the most recent locally-updated version 608 higher than each previous locally-updated version 608. As another particular example, the global parameter updating engine 616 can weight the most common locally-updated version 608 higher than relatively uncommon locally-updated versions 608. As another particular example, the global parameter updating engine 616 can weight the locally-updated version that is least similar to the version stored in the global model parameter store 618 the highest. As another particular example, the global parameter updating engine 616 can weight respective versions based on how many training examples were used to generate them. For example, if a first user device 602a submits a set of locally-updated parameter values 608 that was updated using 100 training examples, and a second user device 602b submits a set of locally-updated parameter values 608 that was updated using 1000 training examples, then the set of value submitted by the second user device 602b can be weighted more heavily.

After determining a set of globally-updated parameter values 620, the global parameter updating engine 616 can store the globally-updated parameter values 620 in the global model parameter store 618, and provide the globally-updated parameter values 620 to each of the N user devices 602a-n. Each user device 602a-n can store the globally-updated parameter values 620 in the respective local model parameter store 610 for future inference calls of the neural network on the user device.

In some implementations, the cloud system 614 can generate and distribute a new set of globally-updated parameter values 620 at regular time intervals. For example, during a given time interval the cloud system 614 can collect and store each set of locally-updated parameter values 608 provided by respective user devices 602a-n; then, the cloud system 614 can generate a batch update to the current parameter values stored in the global model parameter store 618. In some other implementations, the cloud system 614 can generate and distribute a new set of globally-updated parameter values 620 whenever the cloud system 614 receives a new set of locally-updated parameter values 608. In some other implementations, the cloud system 614 can generate and distribute a new set of globally-updated parameter values 620 upon request from a respective user device 602a-n.

Thus, the cloud system 614 can use the user training examples 604 captured by respective user devices 602a-n to improve the performance of the machine learning model, without having direct access to the user training examples 604 themselves. Each user device 602a-n can therefore benefit from the user training examples 604 captured by each other user device 602a-n. The user training examples 604 of the respective user devices 602a-n can also augment the amount of training data available for training the neural network, and greatly improve the diversity of the training data. Each respective user can capture different types of user training examples 604 that the cloud system 614 might otherwise not have had access to, allowing the neural network to benefit from being exposed to a wider variety of network inputs.

In some implementations, the cloud system 614 can determine to generate multiple different sets of globally-updated parameter values 620 corresponding to respective different classes of network inputs. For example, upon receiving multiple different sets of locally-updated parameter values 608 from respective different user devices 602a-n, the cloud system 614 can determine that there is a multi-model distribution of the locally-updated parameter values 608, and determine that each mode of the distribution should define a different version of the neural network. As a particular example, the cloud system 614 can determine to generate a new set of globally-updated parameter values 620 when the number of training examples used to train the current set of globally-updated parameter values 620 increases but the performance (e.g., the testing accuracy) of the current set of globally-updated parameter values 620 is not increasing (e.g., if the performance has plateaued or even decreased). This implies that the new training examples are not improving the model (or even are making the model worse), and therefore that the new training examples might be drawn from a different distribution).

Each different version of the neural network can correspond to one or more respective classes of network inputs that the neural network can receive. For example, if the neural network is an image classification neural network, then the user of a particular user device 602a might generate user training examples 604 that are directed to a very specific type of images, e.g., differentiating images of different species of insect. The user of the particular user device 602a, who might be an expert entomologist, can provide a target output for each training input in the user training examples 604, and generate locally-updated parameter values 608 that are specifically directed to insect classification. The generated locally-updated parameter values 608, however, might not be able to be applied to other classes of image classification tasks; that is, the performance of a neural network having the locally-updated parameter values might decline for every class of image classification task besides the task of classifying images of insects. Therefore, the cloud system 614 can determine to maintain two different sets of global parameter values in the global model parameter store 618: a first set corresponding to the insect classification task, and a second set corresponding to each other image classification task.

Generally, the global model parameter store 618 can store any number of different sets of parameter values corresponding to respective classes of tasks.

In order to identify the type of task to which a set of locally-updated parameter values 608 corresponds, without providing the user training examples 604 used to generate the locally-updated parameter values 608, the respective user device 602a-n can provide a set of statistics characterizing the user training examples 604. For example, the user device 602a-n can identify a distribution of the network outputs generated by the neural network in response to processing the user training examples 604. The cloud system 614 can then use the statistics to determine which set of global parameter values to update in response to receiving the locally-updated parameter values 608 (or, whether to generate a new set of global parameter values according to the received locally-updated parameter values 608).

FIG. 7 shows an example data flow 700 for generating a synaptic connectivity graph 702 and a brain emulation neural network 704 based on the brain 706 of a biological organism. As used throughout this document, a brain may refer to any amount of nervous tissue from a nervous system of a biological organism, and nervous tissue may refer to any tissue that includes neurons (i.e., nerve cells). The biological organism can be, e.g., a worm, a fly, a mouse, a cat, or a human.

An imaging system 708 can be used to generate a synaptic resolution image 710 of the brain 706. An image of the brain 706 may be referred to as having synaptic resolution if it has a spatial resolution that is sufficiently high to enable the identification of at least some synapses in the brain 706. Put another way, an image of the brain 706 may be referred to as having synaptic resolution if it depicts the brain 706 at a magnification level that is sufficiently high to enable the identification of at least some synapses in the brain 706. The image 710 can be a volumetric image, i.e., that characterizes a three-dimensional representation of the brain 706. The image 710 can be represented in any appropriate format, e.g., as a three-dimensional array of numerical values.

The imaging system 708 can be any appropriate system capable of generating synaptic resolution images, e.g., an electron microscopy system. The imaging system 708 can process “thin sections” from the brain 706 (i.e., thin slices of the brain attached to slides) to generate output images that each have a field of view corresponding to a proper subset of a thin section. The imaging system 708 can generate a complete image of each thin section by stitching together the images corresponding to different fields of view of the thin section using any appropriate image stitching technique. The imaging system 708 can generate the volumetric image 710 of the brain by registering and stacking the images of each thin section. Registering two images refers to applying transformation operations (e.g., translation or rotation operations) to one or both of the images to align them. Example techniques for generating a synaptic resolution image of a brain are described with reference to: Z. Zheng, et al., “A complete electron microscopy volume of the brain of adult Drosophila melanogaster,” Cell 174, 730-743 (7018).

A graphing system 712 is configured to process the synaptic resolution image 710 to generate the synaptic connectivity graph 702. The synaptic connectivity graph 702 specifies a set of nodes and a set of edges, such that each edge connects two nodes. To generate the graph 702, the graphing system 712 identifies each neuron in the image 710 as a respective node in the graph, and identifies each synaptic connection between a pair of neurons in the image 710 as an edge between the corresponding pair of nodes in the graph.

The graphing system 712 can identify the neurons and the synapses depicted in the image 710 using any of a variety of techniques. For example, the graphing system 712 can process the image 710 to identify the positions of the neurons depicted in the image 710, and determine whether a synapse connects two neurons based on the proximity of the neurons (as will be described in more detail below). In this example, the graphing system 712 can process an input including: (i) the image, (ii) features derived from the image, or (iii) both, using a machine learning model that is trained using supervised learning techniques to identify neurons in images.

The machine learning model can be, e.g., a convolutional neural network model or a random forest model. The output of the machine learning model can include a neuron probability map that specifies a respective probability that each voxel in the image is included in a neuron. The graphing system 712 can identify contiguous clusters of voxels in the neuron probability map as being neurons.

Optionally, prior to identifying the neurons from the neuron probability map, the graphing system 712 can apply one or more filtering operations to the neuron probability map, e.g., with a Gaussian filtering kernel. Filtering the neuron probability map can reduce the amount of “noise” in the neuron probability map, e.g., where only a single voxel in a region is associated with a high likelihood of being a neuron.

The machine learning model used by the graphing system 712 to generate the neuron probability map can be trained using supervised learning training techniques on a set of training data. The training data can include a set of training examples, where each training example specifies: (i) a training input that can be processed by the machine learning model, and (ii) a target output that should be generated by the machine learning model by processing the training input. For example, the training input can be a synaptic resolution image of a brain, and the target output can be a “label map” that specifies a label for each voxel of the image indicating whether the voxel is included in a neuron. The target outputs of the training examples can be generated by manual annotation, e.g., where a person manually specifies which voxels of a training input are included in neurons.

Example techniques for identifying the positions of neurons depicted in the image 710 using neural networks (in particular, flood-filling neural networks) are described with reference to: P. H. Li et al.: “Automated Reconstruction of a Serial-Section EM Drosophila Brain with Flood-Filling Networks and Local Realignment,” bioRxiv doi:10.1101/605634 (2019). The graphing system 712 can identify the synapses connecting the neurons in the image 710 based on the proximity of the neurons. For example, the graphing system 712 can determine that a first neuron is connected by a synapse to a second neuron based on the area of overlap between: (i) a tolerance region in the image around the first neuron, and (ii) a tolerance region in the image around the second neuron. That is, the graphing system 712 can determine whether the first neuron and the second neuron are connected based on the number of spatial locations (e.g., voxels) that are included in both: (i) the tolerance region around the first neuron, and (ii) the tolerance region around the second neuron. For example, the graphing system 712 can determine that two neurons are connected if the overlap between the tolerance regions around the respective neurons includes at least a predefined number of spatial locations (e.g., one spatial location). A “tolerance region” around a neuron refers to a contiguous region of the image that includes the neuron. For example, the tolerance region around a neuron can be specified as the set of spatial locations in the image that are either: (i) in the interior of the neuron, or (ii) within a predefined distance of the interior of the neuron.

The graphing system 712 can further identify a weight value associated with each edge in the graph 702. For example, the graphing system 712 can identify a weight for an edge connecting two nodes in the graph 702 based on the area of overlap between the tolerance regions around the respective neurons corresponding to the nodes in the image 710. The area of overlap can be measured, e.g., as the number of voxels in the image 710 that are contained in the overlap of the respective tolerance regions around the neurons. The weight for an edge connecting two nodes in the graph 702 may be understood as characterizing the (approximate) strength of the connection between the corresponding neurons in the brain (e.g., the amount of information flow through the synapse connecting the two neurons).

In addition to identifying synapses in the image 710, the graphing system 712 can further determine the direction of each synapse using any appropriate technique. The “direction” of a synapse between two neurons refers to the direction of information flow between the two neurons, e.g., if a first neuron uses a synapse to transmit signals to a second neuron, then the direction of the synapse would point from the first neuron to the second neuron. Example techniques for determining the directions of synapses connecting pairs of neurons are described with reference to: C. Seguin, A. Razi, and A. Zalesky: “Inferring neural signalling directionality from undirected structure connectomes,” Nature Communications 10, 4289 (2019), doi:10.1038/s41467-019-12201-w.

In implementations where the graphing system 712 determines the directions of the synapses in the image 710, the graphing system 712 can associate each edge in the graph 702 with the direction of the corresponding synapse. That is, the graph 702 can be a directed graph. In some other implementations, the graph 702 can be an undirected graph, i.e., where the edges in the graph are not associated with a direction.

The graph 702 can be represented in any of a variety of ways. For example, the graph 702 can be represented as a two-dimensional array of numerical values with a number of rows and columns equal to the number of nodes in the graph. The component of the array at position (i,j) can have value 1 if the graph includes an edge pointing from node i to node j, and value 0 otherwise. In implementations where the graphing system 712 determines a weight value for each edge in the graph 702, the weight values can be similarly represented as a two-dimensional array of numerical values. More specifically, if the graph includes an edge connecting node i to node j, the component of the array at position (i,j) can have a value given by the corresponding edge weight, and otherwise the component of the array at position (i,j) can have value 0.

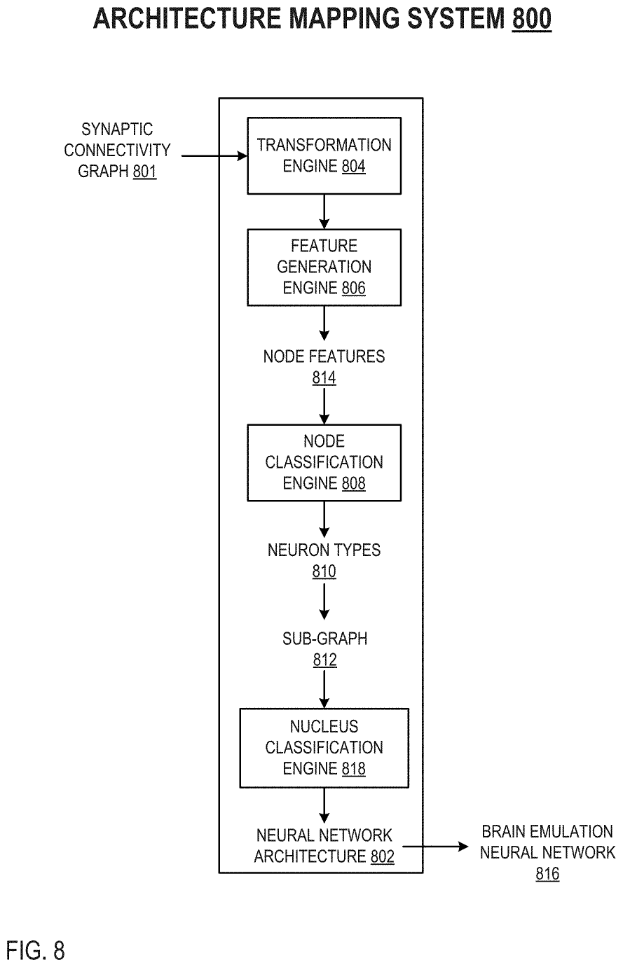

An architecture mapping system 720 can process the synaptic connectivity graph 702 to determine the architecture of the brain emulation neural network 704. For example, the architecture mapping system 720 can map each node in the graph 702 to: (i) an artificial neuron, (ii) a neural network layer, or (iii) a group of neural network layers, in the architecture of the brain emulation neural network 704. The architecture mapping system 720 can further map each edge of the graph 702 to a connection in the brain emulation neural network 704, e.g., such that a first artificial neuron that is connected to a second artificial neuron is configured to provide its output to the second artificial neuron. In some implementations, the architecture mapping system 720 can apply one or more transformation operations to the graph 702 before mapping the nodes and edges of the graph 702 to corresponding components in the architecture of the brain emulation neural network 704, as will be described in more detail below. An example architecture mapping system is described in more detail below with reference to FIG. 8.

The brain emulation neural network 704 can be provided to a training system 714 that trains the brain emulation neural network using machine learning techniques, i.e., generates an update to the respective values of one or more parameters of the brain emulation neural network.

In some implementations, as described above, the brain emulation neural network 704 is a subnetwork of a neural network that includes one or more other neural network layers, e.g., one or more other subnetworks. In some such implementations, the parameter values of the brain emulation neural network 704 are not trained, i.e., are determined according to the synaptic connectivity graph 702. For example, the brain emulation neural network 704 can be a subnetwork of a reservoir computing neural network, e.g., the reservoir computing neural network 202 depicted in FIG. 2. In these implementations, the training system 714 can only generate updates to the parameter values of other trained subnetworks of the neural network, and not to the parameter values of the brain emulation neural network 704.

Although the below description refers to training the brain emulation neural network, it is to be understood that the description can also apply to training a neural network that includes the brain emulation neural network.

In some implementations, the training system 714 is a supervised training system that is configured to train the brain emulation neural network 704 using a set of training data. The training data can include multiple training examples, where each training example specifies: (i) a training input, and (ii) a corresponding target output that should be generated by the brain emulation neural network 704 by processing the training input. In one example, the direct training system 714 can train the brain emulation neural network 704 over multiple training iterations using a gradient descent optimization technique, e.g., stochastic gradient descent. In this example, at each training iteration, the direct training system 714 can sample a “batch” (set) of one or more training examples from the training data, and process the training inputs specified by the training examples to generate corresponding network outputs. The direct training system 714 can evaluate an objective function that measures a similarity between: (i) the target outputs specified by the training examples, and (ii) the network outputs generated by the brain emulation neural network, e.g., a cross-entropy or squared-error objective function. The direct training system 714 can determine gradients of the objective function, e.g., using backpropagation techniques, and update the parameter values of the brain emulation neural network 704 using the gradients, e.g., using any appropriate gradient descent optimization algorithm, e.g., RMSprop or Adam.

In some other implementations, the training system 714 is an adversarial training system that is configured to train the brain emulation neural network 704 in an adversarial fashion. For example, the brain emulation neural network 704 can be configured to generate network outputs that represent realistic data that might have been captured by sensors in the real world, e.g., realistic audio data, images, video frames, or text segments. The training system 714 can include a discriminator neural network that is configured to process network outputs generated by the brain emulation neural network 704 to generate a prediction of whether the network outputs are “real” outputs (i.e., outputs that were not generated by the brain emulation neural network, e.g., outputs that represent data that was captured from the real world) or “synthetic” outputs (i.e., outputs generated by the brain emulation neural network 704). The training system can then determine an update to the parameters of the brain emulation neural network in order to increase an error in the prediction of the discriminator neural network; that is, the goal of the brain emulation neural network is to generate synthetic outputs that are realistic enough that the discriminator neural network predicts them to be real outputs. In some implementations, concurrently with training the brain emulation neural network 704, the training system 714 generates updates to the parameters of the discriminator neural network.

In some other implementations, the training system 714 is a distillation training system that is configured to use the brain emulation neural network 704 to facilitate training of a “student” neural network having a less complex architecture than the brain emulation neural network 704. The complexity of a neural network architecture can be measured, e.g., by the number of parameters required to specify the operations performed by the neural network. The training system 714 can train the student neural network to match the outputs generated by the brain emulation neural network. After training, the student neural network can inherit the capacity of the brain emulation neural network 704 to effectively solve certain tasks, while consuming fewer computational resources (e.g., memory and computing power) than the brain emulation neural network 704. Typically, the training system 714 does not update the parameters of the brain emulation neural network 704 while training the student neural network. That is, in these implementations, the training system 714 is configured to train the student neural network instead of the brain emulation neural network 704.

As a particular example, the training system 714 can be a distillation training system that trains the student neural network in an adversarial manner. For example, the training system 714 can include a discriminator neural network that is configured to process network outputs that were generated either by the brain emulation neural network 704 or the student neural network, and to generate a prediction of whether the network outputs where generated by the brain emulation neural network 704 or the student neural network. The training system can then determine an update to the parameters of the student neural network in order to increase an error in the prediction of the discriminator neural network; that is, the goal of the student neural network is to generate network outputs that resemble network outputs generated by the brain emulation neural network 702 so that the discriminator neural network predicts that they were generated by the brain emulation neural network 704.

After the training system 714 has completed training the brain emulation neural network 704 (or a neural network that includes the brain emulation neural network as a subnetwork), the brain emulation neural network 704 can be deployed by a deployment system 722. That is, the operations of the brain emulation neural network 704 can be implemented on a device or a system of devices for performing inference, i.e., receiving network inputs and processing the network inputs to generate network outputs. In some implementations, the brain emulation neural network 704 can be deployed onto a cloud system, i.e., a distributed computing system having multiple computing nodes, e.g., hundreds or thousands of computing nodes, in one or more locations. In some other implementations, the brain emulation neural network 704 can be deployed onto a user device.

In some implementations, the operations of the brain emulation neural network 704 can be executed using an analog circuit designed according to the network architecture of the brain emulation neural network 704.

FIG. 8 shows an example architecture mapping system 800. The architecture mapping system 800 is an example of a system implemented as computer programs on one or more computers in one or more locations in which the systems, components, and techniques described below are implemented.

The architecture mapping system 800 is configured to process a synaptic connectivity graph 801 (e.g., the synaptic connectivity graph 702 depicted in FIG. 7) to determine a corresponding neural network architecture 802 of a brain emulation neural network 816 (e.g., the brain emulation neural network 704 depicted in FIG. 7). The architecture mapping system 800 can determine the architecture 802 using one or more of: a transformation engine 804, a feature generation engine 806, a node classification engine 808, and a nucleus classification engine 818, which will each be described in more detail next.

The transformation engine 804 can be configured to apply one or more transformation operations to the synaptic connectivity graph 801 that alter the connectivity of the graph 801, i.e., by adding or removing edges from the graph. A few examples of transformation operations follow.

In one example, to apply a transformation operation to the graph 801, the transformation engine 804 can randomly sample a set of node pairs from the graph (i.e., where each node pair specifies a first node and a second node). For example, the transformation engine can sample a predefined number of node pairs in accordance with a uniform probability distribution over the set of possible node pairs. For each sampled node pair, the transformation engine 804 can modify the connectivity between the two nodes in the node pair with a predefined probability (e.g., 0.1%). In one example, the transformation engine 804 can connect the nodes by an edge (i.e., if they are not already connected by an edge) with the predefined probability. In another example, the transformation engine 804 can reverse the direction of any edge connecting the two nodes with the predefined probability. In another example, the transformation engine 804 can invert the connectivity between the two nodes with the predefined probability, i.e., by adding an edge between the nodes if they are not already connected, and by removing the edge between the nodes if they are already connected.

In another example, the transformation engine 804 can apply a convolutional filter to a representation of the graph 801 as a two-dimensional array of numerical values. As described above, the graph 801 can be represented as a two-dimensional array of numerical values where the component of the array at position (i,j) can have value 1 if the graph includes an edge pointing from node i to node j, and value 0 otherwise. The convolutional filter can have any appropriate kernel, e.g., a spherical kernel or a Gaussian kernel. After applying the convolutional filter, the transformation engine 804 can quantize the values in the array representing the graph, e.g., by rounding each value in the array to 0 or 1, to cause the array to unambiguously specify the connectivity of the graph. Applying a convolutional filter to the representation of the graph 801 can have the effect of regularizing the graph, e.g., by smoothing the values in the array representing the graph to reduce the likelihood of a component in the array having a different value than many of its neighbors.

In some cases, the graph 801 can include some inaccuracies in representing the synaptic connectivity in the biological brain. For example, the graph can include nodes that are not connected by an edge despite the corresponding neurons in the brain being connected by a synapse, or “spurious” edges that connect nodes in the graph despite the corresponding neurons in the brain not being connected by a synapse. Inaccuracies in the graph can result, e.g., from imaging artifacts or ambiguities in the synaptic resolution image of the brain that is processed to generate the graph. Regularizing the graph, e.g., by applying a convolutional filter to the representation of the graph, can increase the accuracy with which the graph represents the synaptic connectivity in the brain, e.g., by removing spurious edges.

The architecture mapping system 800 can use the feature generation engine 806 and the node classification engine 808 to determine predicted “types” 810 of the neurons corresponding to the nodes in the graph 801. The type of a neuron can characterize any appropriate aspect of the neuron. In one example, the type of a neuron can characterize the function performed by the neuron in the brain, e.g., a visual function by processing visual data, an olfactory function by processing odor data, or a memory function by retaining information. After identifying the types of the neurons corresponding to the nodes in the graph 801, the architecture mapping system 800 can identify a sub-graph 812 of the overall graph 801 based on the neuron types, and determine the neural network architecture 802 based on the sub-graph 812. The feature generation engine 806 and the node classification engine 808 are described in more detail next.

The feature generation engine 806 can be configured to process the graph 801 (potentially after it has been modified by the transformation engine 804) to generate one or more respective node features 814 corresponding to each node of the graph 801. The node features corresponding to a node can characterize the topology (i.e., connectivity) of the graph relative to the node. In one example, the feature generation engine 806 can generate a node degree feature for each node in the graph 801, where the node degree feature for a given node specifies the number of other nodes that are connected to the given node by an edge. In another example, the feature generation engine 806 can generate a path length feature for each node in the graph 801, where the path length feature for a node specifies the length of the longest path in the graph starting from the node. A path in the graph may refer to a sequence of nodes in the graph, such that each node in the path is connected by an edge to the next node in the path. The length of a path in the graph may refer to the number of nodes in the path. In another example, the feature generation engine 806 can generate a neighborhood size feature for each node in the graph 801, where the neighborhood size feature for a given node specifies the number of other nodes that are connected to the node by a path of length at most N. In this example, N can be a positive integer value. In another example, the feature generation engine 806 can generate an information flow feature for each node in the graph 801. The information flow feature for a given node can specify the fraction of the edges connected to the given node that are outgoing edges, i.e., the fraction of edges connected to the given node that point from the given node to a different node. In some implementations, the feature generation engine 806 can generate one or more node features that do not directly characterize the topology of the graph relative to the nodes. In one example, the feature generation engine 806 can generate a spatial position feature for each node in the graph 801, where the spatial position feature for a given node specifies the spatial position in the brain of the neuron corresponding to the node, e.g., in a Cartesian coordinate system of the synaptic resolution image of the brain. In another example, the feature generation engine 806 can generate a feature for each node in the graph 801 indicating whether the corresponding neuron is excitatory or inhibitory. In another example, the feature generation engine 806 can generate a feature for each node in the graph 801 that identifies the neuropil region associated with the neuron corresponding to the node.

In some cases, the feature generation engine 806 can use weights associated with the edges in the graph in determining the node features 814. As described above, a weight value for an edge connecting two nodes can be determined, e.g., based on the area of any overlap between tolerance regions around the neurons corresponding to the nodes. In one example, the feature generation engine 806 can determine the node degree feature for a given node as a sum of the weights corresponding to the edges that connect the given node to other nodes in the graph. In another example, the feature generation engine 806 can determine the path length feature for a given node as a sum of the edge weights along the longest path in the graph starting from the node.

The node classification engine 808 can be configured to process the node features 814 to identify a predicted neuron type 810 corresponding to certain nodes of the graph 801. In one example, the node classification engine 808 can process the node features 814 to identify a proper subset of the nodes in the graph 801 with the highest values of the path length feature. For example, the node classification engine 808 can identify the nodes with a path length feature value greater than the 90th percentile (or any other appropriate percentile) of the path length feature values of all the nodes in the graph. The node classification engine 808 can then associate the identified nodes having the highest values of the path length feature with the predicted neuron type of “primary sensory neuron.” In another example, the node classification engine 808 can process the node features 814 to identify a proper subset of the nodes in the graph 801 with the highest values of the information flow feature, i.e., indicating that many of the edges connected to the node are outgoing edges. The node classification engine 808 can then associate the identified nodes having the highest values of the information flow feature with the predicted neuron type of “sensory neuron.” In another example, the node classification engine 808 can process the node features 814 to identify a proper subset of the nodes in the graph 801 with the lowest values of the information flow feature, i.e., indicating that many of the edges connected to the node are incoming edges (i.e., edges that point towards the node). The node classification engine 808 can then associate the identified nodes having the lowest values of the information flow feature with the predicted neuron type of “associative neuron.”

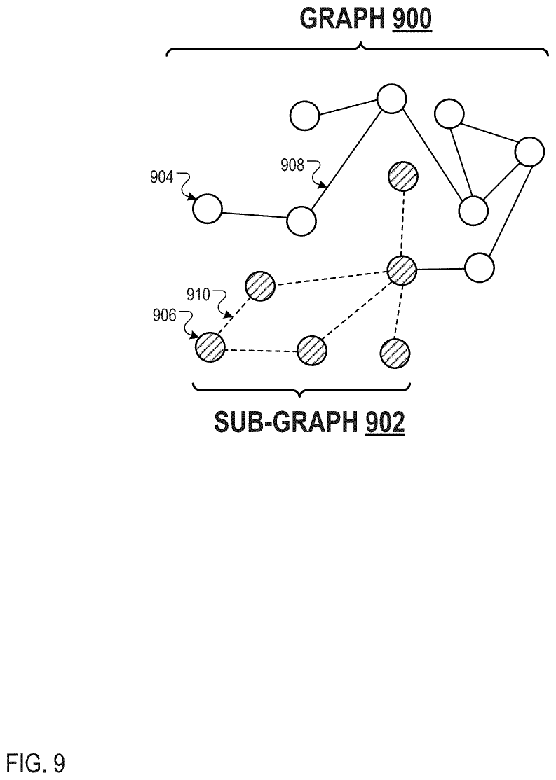

The architecture mapping system 800 can identify a sub-graph 812 of the overall graph 801 based on the predicted neuron types 810 corresponding to the nodes of the graph 801. A “sub-graph” may refer to a graph specified by: (i) a proper subset of the nodes of the graph 801, and (ii) a proper subset of the edges of the graph 801. FIG. 9 provides an illustration of an example sub-graph of an overall graph. In one example, the architecture mapping system 800 can select: (i) each node in the graph 801 corresponding to particular neuron type, and (ii) each edge in the graph 801 that connects nodes in the graph corresponding to the particular neuron type, for inclusion in the sub-graph 812. The neuron type selected for inclusion in the sub-graph can be, e.g., visual neurons, olfactory neurons, memory neurons, or any other appropriate type of neuron. In some cases, the architecture mapping system 800 can select multiple neuron types for inclusion in the sub-graph 812, e.g., both visual neurons and olfactory neurons.

The type of neuron selected for inclusion in the sub-graph 812 can be determined based on the task which the brain emulation neural network 816 will be configured to perform. In one example, the brain emulation neural network 816 can be configured to perform an image processing task, and neurons that are predicted to perform visual functions (i.e., by processing visual data) can be selected for inclusion in the sub-graph 812. In another example, the brain emulation neural network 816 can be configured to perform an odor processing task, and neurons that are predicted to perform odor processing functions (i.e., by processing odor data) can be selected for inclusion in the sub-graph 812. In another example, the brain emulation neural network 816 can be configured to perform an audio processing task, and neurons that are predicted to perform audio processing (i.e., by processing audio data) can be selected for inclusion in the sub-graph 812.

If the edges of the graph 801 are associated with weight values (as described above), then each edge of the sub-graph 812 can be associated with the weight value of the corresponding edge in the graph 801. The sub-graph 812 can be represented, e.g., as a two-dimensional array of numerical values, as described with reference to the graph 801.

Determining the architecture 802 of the brain emulation neural network 816 based on the sub-graph 812 rather than the overall graph 801 can result in the architecture 802 having a reduced complexity, e.g., because the sub-graph 812 has fewer nodes, fewer edges, or both than the graph 801. Reducing the complexity of the architecture 802 can reduce consumption of computational resources (e.g., memory and computing power) by the brain emulation neural network 816, e.g., enabling the brain emulation neural network 816 to be deployed in resource-constrained environments, e.g., mobile devices. Reducing the complexity of the architecture 802 can also facilitate training of the brain emulation neural network 816, e.g., by reducing the amount of training data required to train the brain emulation neural network 816 to achieve an threshold level of performance (e.g., prediction accuracy).

In some cases, the architecture mapping system 800 can further reduce the complexity of the architecture 802 using a nucleus classification engine 818. In particular, the architecture mapping system 800 can process the sub-graph 812 using the nucleus classification engine 818 prior to determining the architecture 802. The nucleus classification engine 818 can be configured to process a representation of the sub-graph 812 as a two-dimensional array of numerical values (as described above) to identify one or more “clusters” in the array.

A cluster in the array representing the sub-graph 812 may refer to a contiguous region of the array such that at least a threshold fraction of the components in the region have a value indicating that an edge exists between the pair of nodes corresponding to the component. In one example, the component of the array in position (i,j) can have value 1 if an edge exists from node i to node j, and value 0 otherwise. In this example, the nucleus classification engine 818 can identify contiguous regions of the array such that at least a threshold fraction of the components in the region have the value 1. The nucleus classification engine 818 can identify clusters in the array representing the sub-graph 812 by processing the array using a blob detection algorithm, e.g., by convolving the array with a Gaussian kernel and then applying the Laplacian operator to the array. After applying the Laplacian operator, the nucleus classification engine 818 can identify each component of the array having a value that satisfies a predefined threshold as being included in a cluster.

Each of the clusters identified in the array representing the sub-graph 812 can correspond to edges connecting a “nucleus” (i.e., group) of related neurons in brain, e.g., a thalamic nucleus, a vestibular nucleus, a dentate nucleus, or a fastigial nucleus. After the nucleus classification engine 818 identifies the clusters in the array representing the sub-graph 812, the architecture mapping system 800 can select one or more of the clusters for inclusion in the sub-graph 812. The architecture mapping system 800 can select the clusters for inclusion in the sub-graph 812 based on respective features associated with each of the clusters. The features associated with a cluster can include, e.g., the number of edges (i.e., components of the array) in the cluster, the average of the node features corresponding to each node that is connected by an edge in the cluster, or both. In one example, the architecture mapping system 800 can select a predefined number of largest clusters (i.e., that include the greatest number of edges) for inclusion in the sub-graph 812.

The architecture mapping system 800 can reduce the sub-graph 812 by removing any edge in the sub-graph 812 that is not included in one of the selected clusters, and then map the reduced sub-graph 812 to a corresponding neural network architecture, as will be described in more detail below. Reducing the sub-graph 812 by restricting it to include only edges that are included in selected clusters can further reduce the complexity of the architecture 802, thereby reducing computational resource consumption by the brain emulation neural network 816 and facilitating training of the brain emulation neural network 816.

The architecture mapping system 800 can determine the architecture 802 of the brain emulation neural network 816 from the sub-graph 812 in any of a variety of ways. For example, the architecture mapping system 800 can map each node in the sub-graph 812 to a corresponding: (i) artificial neuron, (ii) artificial neural network layer, or (iii) group of artificial neural network layers in the architecture 802, as will be described in more detail next.

In one example, the neural network architecture 802 can include: (i) a respective artificial neuron corresponding to each node in the sub-graph 812, and (ii) a respective connection corresponding to each edge in the sub-graph 812. In this example, the sub-graph 812 can be a directed graph, and an edge that points from a first node to a second node in the sub-graph 812 can specify a connection pointing from a corresponding first artificial neuron to a corresponding second artificial neuron in the architecture 802. The connection pointing from the first artificial neuron to the second artificial neuron can indicate that the output of the first artificial neuron should be provided as an input to the second artificial neuron. Each connection in the architecture can be associated with a weight value, e.g., that is specified by the weight value associated with the corresponding edge in the sub-graph. An artificial neuron may refer to a component of the architecture 802 that is configured to receive one or more inputs (e.g., from one or more other artificial neurons), and to process the inputs to generate an output. The inputs to an artificial neuron and the output generated by the artificial neuron can be represented as scalar numerical values. In one example, a given artificial neuron can generate an output b as:

$$b = σ ( {\sum }_{i = 1}^{n} {w}_{i} \cdot {a}_{i} ) \qquad ( 1 )$$

where σ(·) is a non-linear “activation” function (e.g., a sigmoid function or an arctangent function), {aᵢ}ᵢ₌₁ⁿ are the inputs provided to the given artificial neuron, and {wᵢ}ᵢ₌₁ⁿ are the weight values associated with the connections between the given artificial neuron and each of the other artificial neurons that provide an input to the given artificial neuron.

In another example, the sub-graph 812 can be an undirected graph, and the architecture mapping system 800 can map an edge that connects a first node to a second node in the sub-graph 812 to two connections between a corresponding first artificial neuron and a corresponding second artificial neuron in the architecture. In particular, the architecture mapping system 800 can map the edge to: (i) a first connection pointing from the first artificial neuron to the second artificial neuron, and (ii) a second connection pointing from the second artificial neuron to the first artificial neuron.

In another example, the sub-graph 812 can be an undirected graph, and the architecture mapping system can map an edge that connects a first node to a second node in the sub-graph 812 to one connection between a corresponding first artificial neuron and a corresponding second artificial neuron in the architecture. The architecture mapping system 800 can determine the direction of the connection between the first artificial neuron and the second artificial neuron, e.g., by randomly sampling the direction in accordance with a probability distribution over the set of two possible directions.

In some cases, the edges in the sub-graph 812 is not be associated with weight values, and the weight values corresponding to the connections in the architecture 802 can be determined randomly. For example, the weight value corresponding to each connection in the architecture 802 can be randomly sampled from a predetermined probability distribution, e.g., a standard Normal (N (0,1)) probability distribution.

In another example, the neural network architecture 802 can include: (i) a respective artificial neural network layer corresponding to each node in the sub-graph 812, and (ii) a respective connection corresponding to each edge in the sub-graph 812. In this example, a connection pointing from a first layer to a second layer can indicate that the output of the first layer should be provided as an input to the second layer. An artificial neural network layer may refer to a collection of artificial neurons, and the inputs to a layer and the output generated by the layer can be represented as ordered collections of numerical values (e.g., tensors of numerical values). In one example, the architecture 802 can include a respective convolutional neural network layer corresponding to each node in the sub-graph 812, and each given convolutional layer can generate an output d as:

$$d = σ ( {h}_{θ} ( {\sum }_{i = 1}^{n} {w}_{i} \cdot {c}_{i} ) ) \qquad ( 2 )$$

where each cᵢ (i=1, . . . , n) is a tensor (e.g., a two- or three-dimensional array) of numerical values provided as an input to the layer, each wᵢ (i=1, . . . , n) is a weight value associated with the connection between the given layer and each of the other layers that provide an input to the given layer (where the weight value for each edge can be specified by the weight value associated with the corresponding edge in the sub-graph), NO represents the operation of applying one or more convolutional kernels to an input to generate a corresponding output, and σ(·) is a non-linear activation function that is applied element-wise to each component of its input. In this example, each convolutional kernel can be represented as an array of numerical values, e.g., where each component of the array is randomly sampled from a predetermined probability distribution, e.g., a standard Normal probability distribution.

In another example, the architecture mapping system 800 can determine that the neural network architecture includes: (i) a respective group of artificial neural network layers corresponding to each node in the sub-graph 812, and (ii) a respective connection corresponding to each edge in the sub-graph 812. The layers in a group of artificial neural network layers corresponding to a node in the sub-graph 812 can be connected, e.g., as a linear sequence of layers, or in any other appropriate manner.

The neural network architecture 802 can include one or more artificial neurons that are identified as “input” artificial neurons and one or more artificial neurons that are identified as “output” artificial neurons. An input artificial neuron may refer to an artificial neuron that is configured to receive an input from a source that is external to the brain emulation neural network 816. An output artificial neural neuron may refer to an artificial neuron that generates an output which is considered part of the overall output generated by the brain emulation neural network 816. The architecture mapping system 800 can add artificial neurons to the architecture 802 in addition to those specified by nodes in the sub-graph 812 (or the graph 801), and designate the added neurons as input artificial neurons and output artificial neurons. For example, for a brain emulation neural network 816 that is configured to process an input including a 100×100 image to generate an output indicating whether the image is included in each of 1000 categories, the architecture mapping system 800 can add 10,000 (=100×100) input artificial neurons and 1000 output artificial neurons to the architecture. Input and output artificial neurons that are added to the architecture 802 can be connected to the other neurons in the architecture in any of a variety of ways. For example, the input and output artificial neurons can be densely connected to every other neuron in the architecture.

Various operations performed by the described architecture mapping system 800 are optional or can be implemented in a different order. For example, the architecture mapping system 800 can refrain from applying transformation operations to the graph 801 using the transformation engine 804, and refrain from extracting a sub-graph 812 from the graph 801 using the feature generation engine 806, the node classification engine 808, and the nucleus classification engine 818. In this example, the architecture mapping system 800 can directly map the graph 801 to the neural network architecture 802, e.g., by mapping each node in the graph to an artificial neuron and mapping each edge in the graph to a connection in the architecture, as described above.

FIG. 9 illustrates an example graph 900 and an example sub-graph 902. Each node in the graph 900 is represented by a circle (e.g., 904 and 906), and each edge in the graph 900 is represented by a line (e.g., 908 and 910). In this illustration, the graph 900 can be considered a simplified representation of a synaptic connectivity graph (an actual synaptic connectivity graph can have far more nodes and edges than are depicted in FIG. 9). A sub-graph 902 can be identified in the graph 900, where the sub-graph 902 includes a proper subset of the nodes and edges of the graph 900. In this example, the nodes included in the sub-graph 902 are hatched (e.g., 906) and the edges included in sub-graph 902 are dashed (e.g., 910). The nodes included in the sub-graph 902 can correspond to neurons of a particular type, e.g., neurons having a particular function, e.g., olfactory neurons, visual neurons, or memory neurons. The architecture of the brain emulation neural network can be specified by the structure of the entire graph 900, or by the structure of a sub-graph 902, as described above.

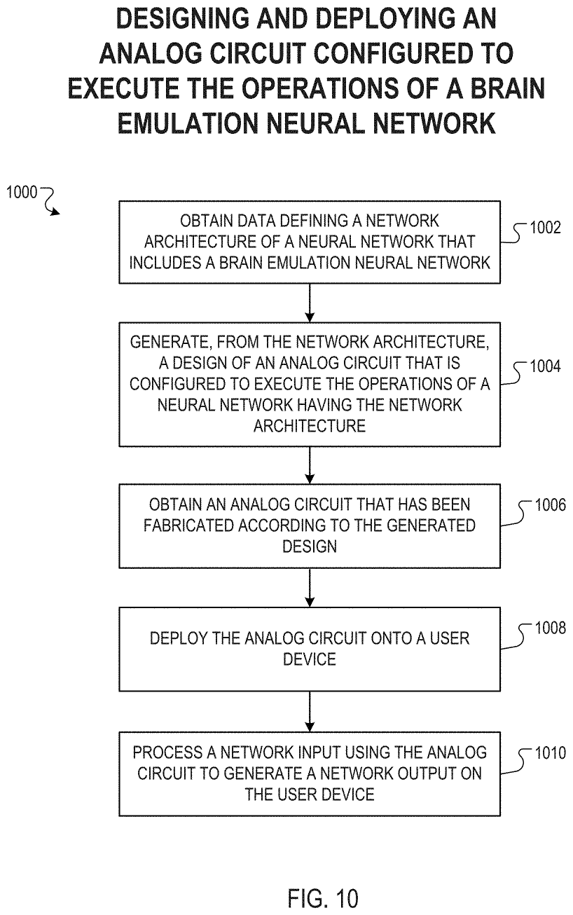

FIG. 10 is a flow diagram of an example process 1000 for designing and deploying an analog circuit configured to execute the operations of a brain emulation neural network. For convenience, the process 1000 will be described as being performed by a system of one or more computers located in one or more locations. For example, an analog circuit design system, e.g., the analog circuit design system 300 of FIG. 3, appropriately programmed in accordance with this specification, can perform the process 1000.

The system obtains data defining a network architecture of a neural network that includes a brain emulation neural network (step 1002). The network architecture can be determined using a graph representing synaptic connectivity between neurons in the brain of a biological organism.

The system generates, from the network architecture, a design of an analog circuit that is configured to execute the operations of a neural network having the network architecture (step 1004). For example, the design of the analog circuit can include a netlist corresponding to the network architecture. In some implementations, the system can generate an initial design using the network architecture and then simplify the analog design by removing one or more components from initial design.

The system obtains an analog circuit that has been fabricated according to the generated design (step 1006). For example, the system can fabricate the analog circuit using the generated design.

The system deploys the analog circuit onto a user device (step 1008). In some implementations, the analog circuit include one or more field-programmable components. In these implementations, the user device can update the values of the field-programmable components using training examples corresponding to the user of the user device.

The system processes a network input using the analog circuit to generate a network output on the user device (step 1010).

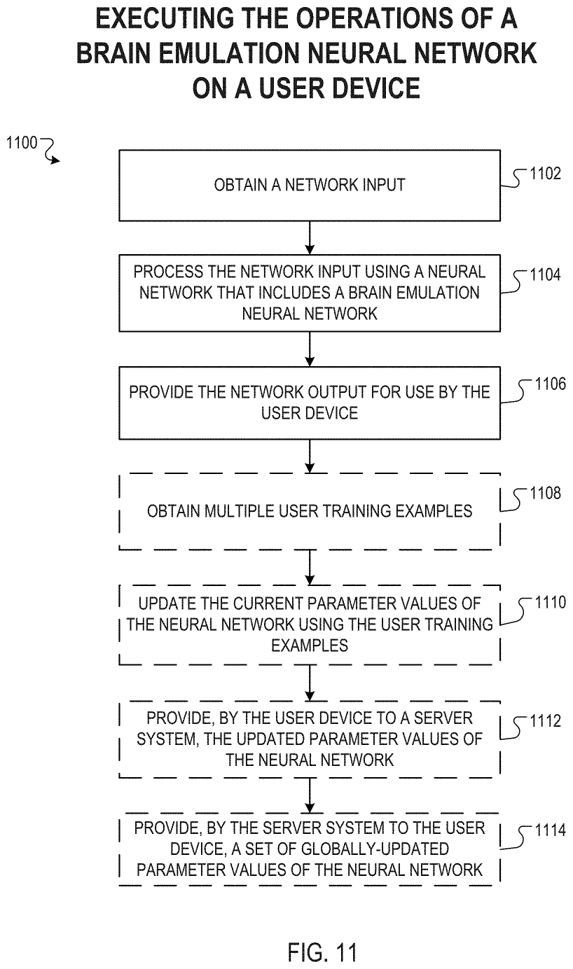

FIG. 11 is a flow diagram of an example process 1100 for executing the operations of a brain emulation neural network on a user device. For convenience, the process 1100 will be described as being performed by a system of one or more computers located in one or more locations. For example, a brain emulation neural network inference system, e.g., the brain emulation neural network inference system 500 of FIG. 5, appropriately programmed in accordance with this specification, can perform the process 1100.

The system obtains a network input (step 1102).

The system processes the network input using a neural network to generate a network output (step 1104). The neural network includes a brain emulation neural network having a network architecture that has been determined using a synaptic connectivity graph.

The system provides the network output for use by the user device (step 1106).

In some implementations, the neural network can include one or more parameters that can be updated by the user device. In these implementations, the system can obtain multiple user training examples (step 1108). Each user training example corresponds to the user of the user device. The system can then update the current parameter values of the neural network using the user training examples (step 1110).

In some implementations, a server system maintains current global parameter values for the neural network. In these implementations, the system can provide, from the user device to a server system, the updated parameter values of the neural network (step 1112). The server system can then use the updated parameter values to update the current global parameter values stored by the server system. In some such implementations, the user device does not provide to the server system the user training examples obtained in step 1108.

In some such implementations, the server system can receive respective updated parameter values from multiple different user devices, and can update the current global parameter values for the neural network using the multiple different sets of updated parameter values to generate a set of globally-updated parameter values. In these implementations, the system can provide, from the server system to the user device, the set of globally-updated parameter values of the neural network (step 1114).

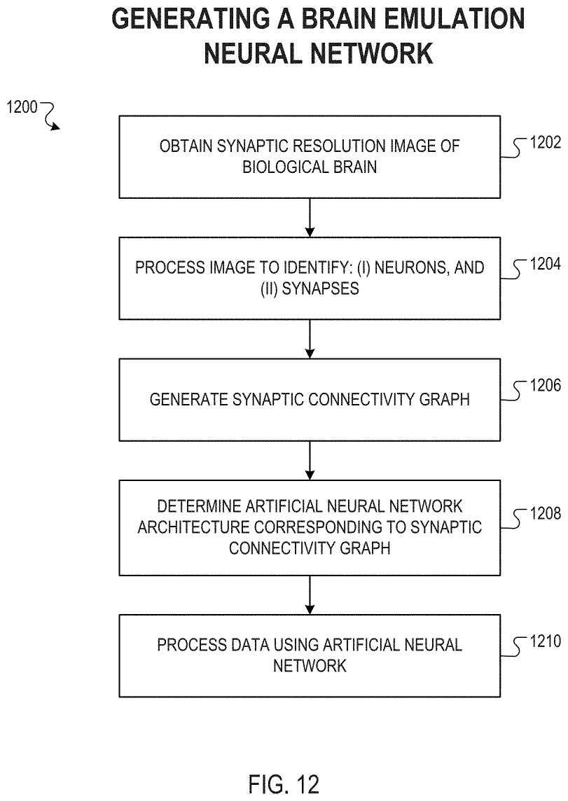

FIG. 12 is a flow diagram of an example process 1200 for generating a brain emulation neural network. For convenience, the process 1200 will be described as being performed by a system of one or more computers located in one or more locations.

The system obtains a synaptic resolution image of at least a portion of a brain of a biological organism (1202).

The system processes the image to identify: (i) neurons in the brain, and (ii) synaptic connections between the neurons in the brain (1204).

The system generates data defining a graph representing synaptic connectivity between the neurons in the brain (1206). The graph includes a set of nodes and a set of edges, where each edge connects a pair of nodes. The system identifies each neuron in the brain as a respective node in the graph, and each synaptic connection between a pair of neurons in the brain as an edge between a corresponding pair of nodes in the graph.

The system determines an artificial neural network architecture corresponding to the graph representing the synaptic connectivity between the neurons in the brain (1208).

The system processes a network input using an artificial neural network having the artificial neural network architecture to generate a network output (1210).

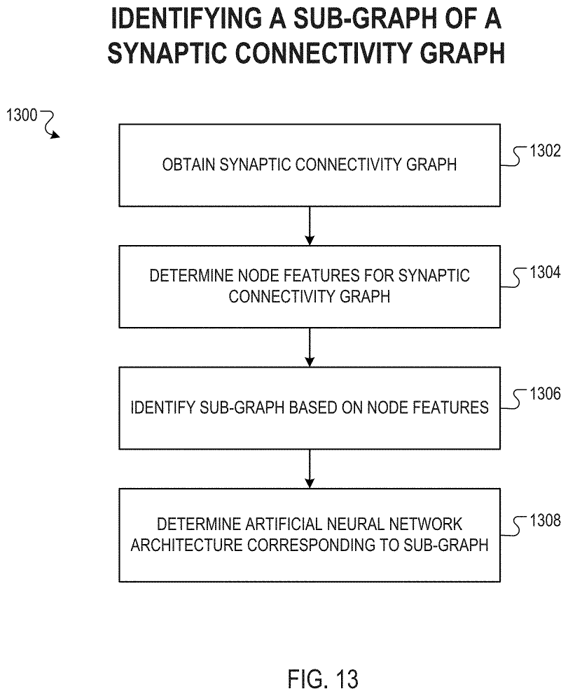

FIG. 13 is a flow diagram of an example process 1300 for determining an artificial neural network architecture corresponding to a sub-graph of a synaptic connectivity graph. For convenience, the process 1300 will be described as being performed by a system of one or more computers located in one or more locations. For example, an architecture mapping system, e.g., the architecture mapping system 800 of FIG. 8, appropriately programmed in accordance with this specification, can perform the process 1300.

The system obtains data defining a graph representing synaptic connectivity between neurons in a brain of a biological organism (1302). The graph includes a set of nodes and edges, where each edge connects a pair of nodes. Each node corresponds to a respective neuron in the brain of the biological organism, and each edge connecting a pair of nodes in the graph corresponds to a synaptic connection between a pair of neurons in the brain of the biological organism.

The system determines, for each node in the graph, a respective set of one or more node features characterizing a structure of the graph relative to the node (1304).

The system identifies a sub-graph of the graph (1306). In particular, the system selects a proper subset of the nodes in the graph for inclusion in the sub-graph based on the node features of the nodes in the graph.

The system determines an artificial neural network architecture corresponding to the sub-graph of the graph (1308).

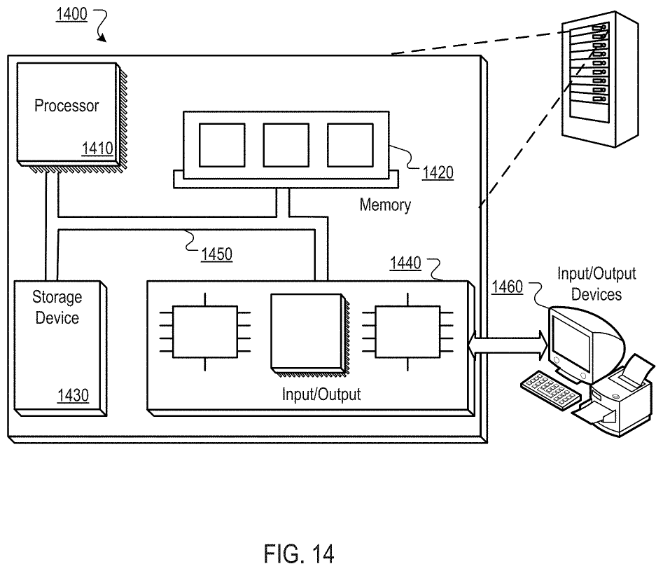

FIG. 14 is a block diagram of an example computer system 1400 that can be used to perform operations described previously. The system 1400 includes a processor 1410, a memory 1420, a storage device 1430, and an input/output device 1440. Each of the components 1410, 1420, 1430, and 1440 can be interconnected, for example, using a system bus 1450. The processor 1410 is capable of processing instructions for execution within the system 1400. In one implementation, the processor 1410 is a single-threaded processor. In another implementation, the processor 1410 is a multi-threaded processor. The processor 1410 is capable of processing instructions stored in the memory 1420 or on the storage device 1430.

The memory 1420 stores information within the system 1400. In one implementation, the memory 1420 is a computer-readable medium. In one implementation, the memory 1420 is a volatile memory unit. In another implementation, the memory 1420 is a non-volatile memory unit.

The storage device 1430 is capable of providing mass storage for the system 1400. In one implementation, the storage device 1430 is a computer-readable medium. In various different implementations, the storage device 1430 can include, for example, a hard disk device, an optical disk device, a storage device that is shared over a network by multiple computing devices (for example, a cloud storage device), or some other large capacity storage device.

The input/output device 1440 provides input/output operations for the system 1400. In one implementation, the input/output device 1440 can include one or more network interface devices, for example, an Ethernet card, a serial communication device, for example, and RS-232 port, and/or a wireless interface device, for example, and 802.11 card. In another implementation, the input/output device 1440 can include driver devices configured to receive input data and send output data to other input/output devices, for example, keyboard, printer and display devices 1460. Other implementations, however, can also be used, such as mobile computing devices, mobile communication devices, and set-top box television client devices. Although an example processing system has been described in FIG. 14, implementations of the subject matter and the functional operations described in this specification can be implemented in other types of digital electronic circuitry, or in computer software, firmware, or hardware, including the structures disclosed in this specification and their structural equivalents, or in combinations of one or more of them.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field-programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program which may also be referred to or described as a program, software, a software application, an app, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

As used in this specification, an “engine,” or “software engine,” refers to a software implemented input/output system that provides an output that is different from the input. An engine can be an encoded block of functionality, such as a library, a platform, a software development kit (“SDK”), or an object. Each engine can be implemented on any appropriate type of computing device, e.g., servers, mobile phones, tablet computers, notebook computers, music players, e-book readers, laptop or desktop computers, PDAs, smart phones, or other stationary or portable devices, that includes one or more processors and computer readable media. Additionally, two or more of the engines may be implemented on the same computing device, or on different computing devices.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and pointing device, e.g, a mouse, trackball, or a presence sensitive display or other surface by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In addition to the embodiments described above, the following embodiments are also innovative:

Embodiment 1 is a user device comprising:

one or more data processing apparatus; and

one or more storage devices communicatively coupled to the one or more data processing apparatus, wherein the one or more storage devices store instructions that, when executed by the one or more data processing apparatus, cause the one or more data processing apparatus to perform operations comprising:

obtaining a network input;

processing the network input using an artificial neural network to generate a network output, wherein the artificial neural network has a network architecture that has been determined according to a synaptic connectivity graph, wherein the synaptic connectivity graph represents synaptic connectivity between neurons in a brain of a biological organism; and providing the network output for use by the user device.

Embodiment 2 is the user device of embodiment 1, wherein processing the network input using the artificial neural network comprises:

quantizing the network input to generate a quantized network input that has a fixed precision that is lower than an original precision of the network input; and

processing the quantized network input using the artificial neural network.

Embodiment 3 is the user device of embodiment 2, wherein a plurality of network parameters of the artificial neural network are expressed and operate at the fixed precision.

Embodiment 4 is the user device of any one of embodiments 1-3, wherein the network architecture comprises:

a first subnetwork comprising a plurality of untrained first network parameters; and

a second subnetwork comprising a plurality of trained second network parameters.

Embodiment 5 is the user device of embodiment 4, wherein determining the artificial neural network architecture comprises generating values for the plurality of first network parameters and the plurality of second network parameters, comprising:

determining initial values for the plurality of second network parameters;

generating values for the plurality of first network parameters using the graph;

obtaining a plurality of training examples; and

processing the plurality of training examples using the artificial neural network according to i) the initial values for the plurality of second network parameters and ii) the values for the plurality of first network parameters to update the initial values for the plurality of second network parameters.

Embodiment 6 is the user device of any one of embodiments 4 or 5, wherein the operations further comprise:

obtaining a plurality of training examples, wherein one or more of the training examples corresponds to a user of the user device; and

updating current values of the second network parameters based on the plurality of training examples.

Embodiment 7 is the user device of embodiment 6, wherein:

a server system maintains global values of the plurality of second network parameters; and

the operations further comprise providing, to the server system, the updated values of the second network parameters.

Embodiment 8 is the user device of embodiment 7, wherein the server system is configured to update the global values of the second network parameters using the received updated values.

Embodiment 9 is the user device of any one of embodiments 7 or 8, wherein the server system is further configured to:

identify a use case corresponding to the plurality of training examples; and

determine parameter values for a new artificial neural network corresponding to the identified use case using the received updated values.

Embodiment 10 is the user device of any one of embodiments 7-9, wherein the user device does not provide the plurality of training examples to the server system.

Embodiment 11 is the user device of any one of embodiments 1-10, wherein the user device is one of:

a smart phone or tablet,

a scientific field device,

an autonomous vehicle, or

a drone.

Embodiment 12 is the user device of any one of embodiments 1-11, wherein processing the network input using the artificial neural network comprises processing the network input using an analog circuit that has been configured to execute a plurality of operations of the artificial neural network.

Embodiment 13 is the user device of any one of embodiments 1-12, wherein:

the synaptic connectivity graph comprises a plurality of nodes and edges, wherein each edge connects a pair of nodes; and

the synaptic connectivity graph was generated by:
determining a plurality of neurons in the brain of the biological organism and a plurality of synaptic connections between pairs of neurons in the brain of the biological organism;
mapping each neuron in the brain of the biological organism to a respective node in the synaptic connectivity graph; and
mapping each synaptic connection between a pair of neurons in the brain to an edge between a corresponding pair of nodes in the synaptic connectivity graph.

Embodiment 14 is the user device of embodiment 13, wherein determining the plurality of neurons and the plurality of synaptic connections comprises:

obtaining a synaptic resolution image of at least a portion of the brain of the biological organism; and

processing the image to identify the plurality of neurons and the plurality of synaptic connections.

Embodiment 15 is the user device of embodiment 14, wherein determining the network architecture comprises:

mapping each node in the synaptic connectivity graph to a corresponding artificial neuron in the network architecture; and

for each edge in the synaptic connectivity graph:
mapping the edge to a connection between a pair of artificial neurons in the network architecture that correspond to the pair of nodes in the synaptic connectivity graph that are connected by the edge.

Embodiment 16 is the user device of embodiment 15, wherein:

determining the network architecture further comprises processing the image to identify a respective direction of each of the synaptic connections between pairs of neurons in the brain;

generating the synaptic connectivity graph further comprises determining a direction of each edge in the synaptic connectivity graph based on the direction of the synaptic connection corresponding to the edge; and

each connection between a pair of artificial neurons in the network architecture has a direction specified by the direction of the corresponding edge in the synaptic connectivity graph.

Embodiment 17 is the user device of any one of embodiment 15 or 16, wherein:

determining the network architecture further comprises processing the image to determine a respective weight value for each of the synaptic connections between pairs of neurons in the brain;

generating the synaptic connectivity graph further comprises determining a weight value for each edge in the synaptic connectivity graph based on the weight value for the synaptic connection corresponding to the edge; and

each connection between a pair of artificial neurons in the network architecture has a weight value specified by the weight value of the corresponding edge in the synaptic connectivity graph.

Embodiment 18 is a method comprising the operations of any one of embodiments 1-17.

Embodiment 19 is a system comprising: one or more computers and one or more storage devices storing instructions that are operable, when executed by the one or more computers, to cause the one or more computers to perform the operations of any one of embodiments 1 to 17.

Embodiment 20 is one or more non-transitory computer storage media encoded with a computer program, the program comprising instructions that are operable, when executed by data processing apparatus, to cause the data processing apparatus to perform the operations of any one of embodiments 1 to 17.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain some cases, multitasking and parallel processing may be advantageous.

What is claimed is:

1. A user device comprising:
one or more data processing apparatus; and
one or more storage devices communicatively coupled to the one or more data processing apparatus, wherein the one or more storage devices store instructions that, when executed by the one or more data processing apparatus, cause the one or more data processing apparatus to perform operations comprising:
obtaining a network input;
processing the network input using an artificial neural network to generate a network output, wherein the artificial neural network has a network architecture that has been determined according to a synaptic connectivity graph, wherein the synaptic connectivity graph represents synaptic connectivity between neurons in a brain of a biological organism; and
providing the network output for use by the user device.

2. The user device of claim 1, wherein processing the network input using the artificial neural network comprises:
quantizing the network input to generate a quantized network input that has a fixed precision that is lower than an original precision of the network input; and
processing the quantized network input using the artificial neural network.

3. The user device of claim 2, wherein a plurality of network parameters of the artificial neural network are expressed and operate at the fixed precision.

4. The user device of claim 1, wherein the network architecture comprises:
a first subnetwork comprising a plurality of untrained first network parameters; and
a second subnetwork comprising a plurality of trained second network parameters.

5. The user device of claim 4, wherein determining the artificial neural network architecture comprises generating values for the plurality of first network parameters and the plurality of second network parameters, comprising:
determining initial values for the plurality of second network parameters;
generating values for the plurality of first network parameters using the graph;
obtaining a plurality of training examples; and
processing the plurality of training examples using the artificial neural network according to i) the initial values for the plurality of second network parameters and ii) the values for the plurality of first network parameters to update the initial values for the plurality of second network parameters.

6. The user device of claim 4, wherein the operations further comprise:
obtaining a plurality of training examples, wherein one or more of the training examples corresponds to a user of the user device; and
updating current values of the second network parameters based on the plurality of training examples.

7. The user device of claim 6, wherein:
a server system maintains global values of the plurality of second network parameters; and
the operations further comprise providing, to the server system, the updated values of the second network parameters.

8. The user device of claim 7, wherein the server system is configured to update the global values of the second network parameters using the received updated values.

9. The user device of claim 7, wherein the server system is configured to:
identify a use case corresponding to the plurality of training examples; and
determine parameter values for a new artificial neural network corresponding to the identified use case using the received updated values.

10. The user device of claim 7, wherein the user device does not provide the plurality of training examples to the server system.

11. The user device of claim 1, wherein the user device is one of:
a smart phone or tablet,
a scientific field device,
an autonomous vehicle, or
a drone.

12. The user device of claim 1, wherein processing the network input using the artificial neural network comprises processing the network input using an analog circuit that has been configured to execute a plurality of operations of the artificial neural network.

13. The user device of claim 1, wherein:
the synaptic connectivity graph comprises a plurality of nodes and edges, wherein each edge connects a pair of nodes; and
the synaptic connectivity graph was generated by:
determining a plurality of neurons in the brain of the biological organism and a plurality of synaptic connections between pairs of neurons in the brain of the biological organism;
mapping each neuron in the brain of the biological organism to a respective node in the synaptic connectivity graph; and
mapping each synaptic connection between a pair of neurons in the brain to an edge between a corresponding pair of nodes in the synaptic connectivity graph.

14. The user device of claim 13, wherein determining the plurality of neurons and the plurality of synaptic connections comprises:
obtaining a synaptic resolution image of at least a portion of the brain of the biological organism; and
processing the image to identify the plurality of neurons and the plurality of synaptic connections.

15. The user device of claim 14, wherein determining the network architecture comprises:
mapping each node in the synaptic connectivity graph to a corresponding artificial neuron in the network architecture; and
for each edge in the synaptic connectivity graph:
mapping the edge to a connection between a pair of artificial neurons in the network architecture that correspond to the pair of nodes in the synaptic connectivity graph that are connected by the edge.

16. The user device of claim 15, wherein:
determining the network architecture further comprises processing the image to identify a respective direction of each of the synaptic connections between pairs of neurons in the brain;
generating the synaptic connectivity graph further comprises determining a direction of each edge in the synaptic connectivity graph based on the direction of the synaptic connection corresponding to the edge; and
each connection between a pair of artificial neurons in the network architecture has a direction specified by the direction of the corresponding edge in the synaptic connectivity graph.

17. The user device of claim 15, wherein:
determining the network architecture further comprises processing the image to determine a respective weight value for each of the synaptic connections between pairs of neurons in the brain;
generating the synaptic connectivity graph further comprises determining a weight value for each edge in the synaptic connectivity graph based on the weight value for the synaptic connection corresponding to the edge; and
each connection between a pair of artificial neurons in the network architecture has a weight value specified by the weight value of the corresponding edge in the synaptic connectivity graph.

18. A method comprising:
obtaining, by a first component of a user device, a network input;
processing, by the first component of the user device, the network input using an artificial neural network to generate a network output, wherein the artificial neural network has a network architecture that has been determined according to a synaptic connectivity graph, wherein the synaptic connectivity graph represents synaptic connectivity between neurons in a brain of a biological organism; and
providing the network output for use by one or more second components of the user device.

19. The method of claim 18, wherein processing the network input using the artificial neural network comprises processing the network input using an analog circuit that has been configured to execute a plurality of operations of the artificial neural network.

20. One or more non-transitory storage media storing instructions that when executed by one or more computers cause the one or more computers to perform operations comprising:
obtaining, by a first component of a user device, a network input;
processing, by the first component of the user device, the network input using an artificial neural network to generate a network output, wherein the artificial neural network has a network architecture that has been determined according to a synaptic connectivity graph, wherein the synaptic connectivity graph represents synaptic connectivity between neurons in a brain of a biological organism; and
providing the network output for use by one or more second components of the user device.
